(19) United States
(12) Patent Application Publication
(10) Pub. No.: US 2024/0352396 A1
(43) Pub. Date: Oct. 24, 2024

(54) PERICELLULAR OXYGEN-CONTROLLING CELL CULTURE INCUBATOR

(71) Applicant: Northeastern University, Boston, MA (US)

(72) Inventors: Zachary Rogers, Jamaica Plain, MA (US); Thibault Colombani, Boston, MA (US); Sidi A. Bencherif, Boston, MA (US)

(21) Appl. No.: 18/635,442

(22) Filed: Apr. 15, 2024

Related U.S. Application Data
(60) Provisional application No. 63/543,412, filed on Oct. 10, 2023.
(60) Provisional application No. 63/459,059, filed on Apr. 13, 2023.

(51) Int. Cl. C12M 1/34; C12M 1/00

(52) U.S. Cl. CPC C12M 41/34; C12M 41/14

(57) ABSTRACT

Disclosed are methods of culturing cells. The methods may comprise providing the cells and a culture incubator comprising a cell culture container; and an inflow of gas, wherein the cells are contained in the cell culture container. The methods may further comprise providing a continuous inflow of a cell culture gas to the culture incubator, incubating the cells in the culture incubator, monitoring the pericellular oxygen concentration of the cells in the cell culture container, and adjusting the inflow of the cell culture gas, thereby adjusting the pericellular oxygen concentration to a target oxygen concentration. Disclosed are culture incubators, comprising a cell culture container; and an inflow of gas, wherein cells are contained in the cell culture container.

### RELATED APPLICATIONS

This application claims the benefit of priority to U.S. Provisional Patent application Ser. No. 63/459,059, filed Apr. 13, 2023; and U.S. Provisional Patent application Ser. No. 63/543,412, filed Oct. 10, 2023.

### GOVERNMENT SUPPORT

This invention was made with government support under Grant No. 2141019 awarded by the National Science Foundation. The government has certain rights in the invention.

### BACKGROUND

Cell culture is one of the most useful techniques in biomedical research, in which scientists attempt to grow cells in conditions that mimic the body. Oxygen, a critical factor for cell behavior and physiology, is often not controlled because it is very difficult to do so. To address this, there are oxygen-controlling technologies on the market that maintain desired oxygen levels for cells by flushing the surrounding gas phase with nitrogen. Supraphysiological O₂ concentrations (hyperoxia) lead to excessive reactive oxygen species (ROS) production, resulting in cellular damage and dysregulated signaling. It is therefore not surprising that cells grown in physioxia experience less oxidative stress. To address these concerns, tools to perform physiological cell culture have been developed and are commercially available. These products, including portable chambers, tri-gas incubators, and hypoxic workstations, consist of chambers that control O₂ in the atmosphere of cultured cells by adding compressed nitrogen. However, widespread adoption has been hampered by cost, laboratory space requirements, technical challenges, and rapid reoxygenation of cultures. Therefore, there is a critical need to novel methods of controlling pericellular oxygen while culturing cells.

### SUMMARY

In one aspect, described herein are methods of culturing cells. The methods may comprise providing the cells and a culture incubator comprising a cell culture container; and an inflow of gas, wherein the cells are contained in the cell culture container. The methods may further comprise providing a continuous inflow of a cell culture gas to the culture incubator, incubating the cells in the culture incubator, monitoring the pericellular oxygen concentration of the cells in the cell culture container, and adjusting the inflow of the cell culture gas, thereby adjusting the pericellular oxygen concentration to a target oxygen concentration.

In some embodiments, the cell culture container further comprises an oxygen sensor for measuring the pericellular oxygen concentration. In some embodiments, the oxygen sensor is located at the bottom of the cell culture container. For example, the oxygen sensor is the SDR SensorDish® Reader, which is a small 24-channel reader for non-invasive detection of oxygen and pH in multidishes (SensorDishes®). These contain a sensor spot at the bottom of each well. They are read out non-invasively through the transparent bottom. SensorDishes® for oxygen (OxoDish®) and pH (HydroDish®) are available in 24-well and 6-well format. 24-well deep well plates with integrated oxygen (OxoDish®-DW) and pH sensor (HydroDish®-DW) allow measurements in shaken cultures. Read out of oxygen sensors integrated in glass vessels for respiration monitoring is also possible.

The Oxygen sensor may be a Pyroscience® OXSP5 oxygen sensor. Oxygen sensor spots allow oxygen measurements in closed sample containers with contactless read-out through a transparent window (glass, acryl glass) with special adapters and optical fibers for all types of fiber-optic meters from PyroScience®. This enables incubations in closed systems, preventing potential contamination and simplifying oxygen monitoring without sampling, also in combination with pH and temperature sensor spots.

In some embodiments, the pericellular oxygen concentration is measured using a mathematically modeled equation that predicts pericellular oxygen tension in an oxygen-controlled cell culture. In some embodiments, the mathematically modeled equation is Michaelis-Menten kinetics. In some embodiments, the cell culture gas is nitrogen, argon, carbon dioxide, oxygen, and/or ambient air. In some embodiments, the culture incubator comprises a humidity sensor, a carbon dioxide sensor, a temperature sensor, and/or an oxygen sensor. In some embodiments, the culture incubator further comprises an inlet with a mass flow controller for carbon dioxide and/or a mass flow controller for nitrogen. In some embodiments, the culture incubator further comprises an outlet that allows ambient air into the incubator.

In some embodiments, the cells are eukaryotic cells or prokaryotic cells. In some embodiments, the cells are eukaryotic cells; and the eukaryotic cells are cell strains derived from an animal, a plant, or an insect, a primary culture product, or a fungus. In some embodiments, the cells are prokaryotic cells; and the prokaryotic cells are bacteria including Escherichia coli, Bacillus subtilis, cyanobacteria, Actinomycetes methanogen, extreme halophile, or hyperthermophile.

In some embodiments, the target oxygen concentration is for anoxic conditions. In some embodiments, the target oxygen concentration is 0% to about 0.5% oxygen. In some embodiments, the target oxygen concentration is 0% oxygen, about 0.1% oxygen, about 0.2% oxygen, about 0.3% oxygen, about 0.4% oxygen, or about 0.5% oxygen. In some embodiments, the target oxygen concentration is for hypoxic conditions. In some embodiments, the target oxygen concentration is about 0.5% to about 1% oxygen. In some embodiments, the target oxygen concentration is about 0.5% oxygen, about 0.6% oxygen, about 0.7% oxygen, about 0.8% oxygen, about 0.9% oxygen, or about 1% oxygen. In some embodiments, the target oxygen concentration is for physioxic conditions. In some embodiments, the target oxygen concentration is about 2% to about 5% oxygen. In some embodiments, the target oxygen concentration is about 2% oxygen, about 2.5% oxygen, about 3% oxygen, about 3.5% oxygen, about 4% oxygen, about 4.5% oxygen, or about 5% oxygen. In some embodiments, the culture incubator is a bioreactor. In some embodiments, the cell culture container is a 6-well plate, 12-well plate, a 24-well plate, a 96-well plate, a T25 flask, a T75 flask, a T175flask, or a T225 flask.

In one aspect, described herein are culture incubators. The culture incubators may comprise a cell culture container; and an inflow of gas, wherein cells are contained in the cell culture container. The culture incubators may further comprise an oxygen sensor to monitor the pericellular oxygen concentration of the cells in the cell culture container. In some embodiments, the pericellular oxygen concentration is adjusted to a target oxygen concentration by adjusting the inflow of a cell culture gas.

In some embodiments, the oxygen sensor is located at the bottom of the cell culture container. In some embodiments, the pericellular oxygen concentration is measured using a mathematically modeled equation that predict pericellular oxygen tension in an oxygen-controlled cell culture. In some embodiments, the mathematically modeled equation is Michaelis-Menten kinetics. In some embodiments, the cell culture gas is nitrogen, argon, carbon dioxide, oxygen, and/or ambient air. In some embodiments, the culture incubator further comprises a humidity sensor, a carbon dioxide sensor, a temperature sensor, and/or an oxygen sensor. In some embodiments, the culture incubator further comprises an inlet with a mass flow controller for carbon dioxide and/or a mass flow controller for nitrogen.

In some embodiments, the culture incubator comprises an outlet that allows ambient air into the incubator. In some embodiments, the cells are eukaryotic cells or prokaryotic cells. In some embodiments, the cells are eukaryotic cells; and the eukaryotic cells are cell strains derived from an animal, a plant, or an insect, a primary culture product, or a fungus. In some embodiments, the cells are prokaryotic cells; and the prokaryotic cells are bacteria including Escherichia coli, Bacillus subtilis, cyanobacteria, Actinomycetes methanogen, extreme halophile, or hyperthermophile. In some embodiments, the target oxygen concentration is for anoxic conditions.

In some embodiments, the target oxygen concentration is 0% to about 0.5% oxygen. In some embodiments, the target oxygen concentration is 0% oxygen, about 0.1% oxygen, about 0.2% oxygen, about 0.3% oxygen, about 0.4% oxygen, or about 0.5% oxygen. In some embodiments, the target oxygen concentration is for hypoxic conditions. In some embodiments, the target oxygen concentration is about 0.5% to about 1% oxygen. In some embodiments, the target oxygen concentration is about 0.5% oxygen, about 0.6% oxygen, about 0.7% oxygen, about 0.8% oxygen, about 0.9% oxygen, or about 1% oxygen. In some embodiments, the target oxygen concentration is for physioxic conditions. In some embodiments, the target oxygen concentration is about 2% to about 5% oxygen. In some embodiments, the target oxygen concentration is about 2% oxygen, about 2.5% oxygen, about 3% oxygen, about 3.5% oxygen, about 4% oxygen, about 4.5% oxygen, or about 5% oxygen. In some embodiments, the culture incubator is a bioreactor. In some embodiments, the cell culture container is a 6-well plate, 12-well plate, a 24-well plate, a 96-well plate, a T25 flask, a T75 flask, a T175flask, or a T225 flask.

### BRIEF DESCRIPTION OF THE DRAWINGS

FIGS. 1A-1F show that cell culture parameters influence HIF stabilization kinetics in hypoxic (1% (2) culture. FIG. 1A shows O₂ kinetics (left) and time to 1% O₂ (right) of normoxic media (EMEM+10% FBS+1% P/S) placed in a 1% O₂ incubator. FIG. 1B shows O₂ kinetics (left) and time to 10% O₂ (right) of 1% O₂ media placed into different culture vessels under normoxia. FIG. 1C shows O₂ kinetics of MCF7 cultures with varying cell densities (left), medium volumes (middle), and culture vessel type (right) placed in a 1% O₂ incubator. FIG. 1D shows time to 1% O₂ from (C). FIG. 1E shows average fluorescence (Image-iT™ Hypoxia) per cell (left) and representative confocal images at 4 hours (right) for MCF7 cultures (60 mm dish) with 5 or 15 mL of media placed in a 1% O₂ chamber. Red=Image-iT™ Hypoxia as an indicator of cellular hypoxia. FIG. 1F shows percentage of GFP+ cells (left) and representative confocal images at 3 days (right) from MCF7 HIF reporter cells cultured at 7,000 or 29,000 cells/cm² for 4 days in a 1% O₂ incubator. Blue=nuclei stained with DAPI, Green=GFP+ (HIF+) cells. FIG. 1G shows percentage of GFP+ cells (left), GFP mean fluorescence intensity (MFI) (middle) and representative contour plots with outliers (right) for MDA-MB-231 HIF reporter cells cultured in a 24-well plate or T25 flask for up to 5 days in a 1% O₂ incubator. Data were analyzed with ANOVA and Geisser-Greenhouse (FIG. 1E) or Bonferroni (FIGS. 1F-1G) corrections. N=3-4 biological replicates per condition.

FIGS. 2A-2H show that cellular O₂ consumption regularly induces anoxia (0% O₂) in both physioxic (5% O₂) and hypoxic (1% O₂) culture. FIGS. 2A-2B show O₂ profiles (left) and average O₂ tension values (right) of media, human mammary epithelial, MCF7, or MDA-MB-231 cultures seeded onto a 24-well plate in a 5% O₂ (Figure A) or 1% O₂ (Figure B) incubator for 72 hours. FIG. 2C shows O₂ profiles (left) and average O₂ tension values (right) of human dendritic cell (DC) 96-well cultures in a 5% or 1% O₂ incubator for 72 hours. FIG. 2D shows O₂ kinetics of media (green) or MDA-MBA-231 cultures (blue) of the top (dark) or bottom (light) of the well in a 1% O₂ incubator. FIG. 2E shows O₂ profiles of MDA-MB-231 cultures measured at the bottom of the well (spot) or 1 mm above the bottom of the well (needle) in a 1% O₂ incubator. FIG. 2F shows effect of sodium azide (NaN₃) on MCF7 pericellular O₂ tension and cytotoxicity. O₂ profiles (left) and average O₂ tension values (middle) of MCF7 cultures spiked with either 5 mM NaN₃ or PBS in a 1% O₂ incubator. MCF7 cell viability after±incubation with 5 mM NaN₃ for 6 hours in a 1% O₂ incubator (right). FIG. 2G shows average O₂ tension values of media and MCF7 cultures with different cell densities and culture vessel types in a 5% O₂ (left) or 1% O₂ incubator (right) for 72 hours. FIG. 2H shows live, attached (left) and detached (right) cell counts for MCF7 cultures in a 18.6%, 5% or 1% O₂ incubator for 6 days. Data were analyzed with two-tailed t test (F) or ANOVA and Dunnett's (A-B) or Tukey's (C, F, G, H) corrections. N=3-4 biological replicates per condition. Colored * indicates comparison to the control.

FIGS. 3A-3I show that setting the incubator to physiological O₂ conditions mimics ischemia-reperfusion injury in human hepatocyte culture. FIG. 3A shows schematic of the primary human hepatocyte culture. RNA-seq was performed on uncultured hepatocytes and cells cultured in normoxic (18.6% (2) or physioxic (6% O₂) incubator after 36 hours. The physioxic setpoint was chosen based on the O₂ tension found in human liver parenchyma. FIGS. 3B-3C show average O₂ tension values (FIG. 3B) and detached cells per well (FIG. 3C) during each step of the culturing process. FIG. 3D shows principal component analysis (PCA) of the RNA-seq results for normoxic and physioxic cultured hepatocytes. FIG. 3E shows volcano plot indicating upregulated (right) and downregulated (left) genes for physioxic vs. normoxic cultured hepatocytes (p_{adj}<0.05 and |log₂FC|≥1). FIG. 3F shows hypoxia gene set enrichment analysis (GSEA) from the Hallmark database for normoxic vs. uncultured (left) and physioxic vs. uncultured (right). FIGS. 3G-3I show enriched pathways from the Gene Ontology (GO) database for physioxic vs. normoxic samples associated with oxidative stress (FIG. 3G), sterile inflammatory response (FIG. 3H) and mitochondrial and ribosomal biogenesis (FIG. 3I). Data were analyzed with ANOVA and Tukey's correction (B-C). N=3 biological samples per condition for RNA seq analysis.

FIGS. 4A-4E show developing a reaction-diffusion model to predict pericellular O₂ tension in cell cultures. FIG. 4A shows numerical (num) (dashed) and experimental (exp) (solid) O₂ kinetics of normoxic media (EMEM+10% FBS+1% P/S) in different culture vessels placed in a 1% O₂ incubator. FIG. 4B shows diffusion model predictions for different volumes of normoxic media in a 6-well plate (left) or a 24-well plate (right) placed in a 1% O₂ incubator. FIG. 4C shows numerical and experimental O₂ kinetics of MDA-MB-231 cultures seeded in a 24-well plate placed in a 1%, 4%, 6%, or 8% O₂ incubator for 48 hours. FIG. 4D shows reaction-diffusion model predictions of the gradient within MDA-MB-231 cultures seeded in a 24-well plate in a 5% O₂ (left) or 1% O₂ (right) incubator. FIG. 4E shows reaction-diffusion model predictions of MDA-MB-231 cells cultured at different cell densities and culture vessel types in a 5% O₂ incubator. N=4 biological replicates per condition for experimental data.

FIGS. 5A-5I show that pericellular anoxia induces stronger metabolic reprogramming and distinct transcriptional HIFA and HRE controlled gene responses compared to pericellular hypoxia in MCF7 cells. FIG. 5A shows average pericellular O₂ tensions in MCF7 cultures placed in 18.6%, 4.5%, and 1% O₂ incubators for 72 hours. FIGS. 5B-5E shows extracellular concentrations of glucose (FIG. 5B), glutamine (FIG. 5C), lactate (FIG. 5D), and glutamate (FIG. 5E) in different O₂ tensions for 72 hours. Normalized metabolite concentrations over time (left) and metabolite consumption or production rates for 72 hours (right). FIGS. 5F-5I shows gene expression levels of genes associated with the hypoxic response (FIG. 5F), metabolic reprogramming (FIG. 5G), autophagy (FIG. 5H), and immunosuppression (FIG. 5I) in different O₂ tensions for 72 hours. Data were analyzed using ANOVA with Tukey's correction. N=4 biological samples per condition.

FIGS. 6A-6E show oroteomic characterization of pericellular hypoxic and anoxic metabolic reprogramming in 4T1 cells. FIG. 6A shows protein set enrichment analysis (PSEA) using the Hallmark database of mRNA processing and protein translation pathways for Hypoxia (H) vs. Normoxia (N), Anoxia (A) vs. Normoxia, and Hypoxia vs. Anoxia. FIG. 6B shows PSEA using the Hallmark database of hypoxic response pathways (left). Heat maps of selected proteins in Hallmark hypoxia, epithelial to mesenchymal transition (EMT), and reactive O₂ species (ROS) pathways (right) (Hypoxia vs. Anoxia). FIG. 6C shows heat map of glycolytic and hypoxic response proteins. FIG. 6D shows PSEA using Reactome database for fatty acid (FA) metabolism pathways for Hypoxia vs. Anoxia (top). Heat maps of selected proteins in fatty acid β-oxidation and synthesis Reactome pathways (bottom). FIG. 6E shows heat maps of selected proteins from Tricarboxylic acid (TCA) cycle and electron transport chain (ETC) processes for Hypoxia vs. Normoxia and Anoxia vs. Normoxia. PSEA using the Reactome database of TCA and ETC pathways for Hypoxia vs. Normoxia, Anoxia vs. Normoxia (left), and Hypoxia vs. Anoxia (right). NES=normalized enrichment score.

FIG. 7 shows that medium volume influences cellular hypoxia kinetics in 1% O₂ culture. Representative confocal images of MCF7 cultures in 60 mm dishes with 5 mL or 15 mL of medium placed inside a 1% O₂ chamber for 12 hours. Confocal images were taken every 30 min. Image-iT™ Hypoxia as an indicator of cellular hypoxia. Nuclei stained with DAPI. Cytoplasm stained with Celltracker. N=3 biological replicates per condition.

FIG. 8 shows that cell density influences HIF stabilization kinetics in 1% O₂ culture. Representative confocal images of MCF7 HIF reporter cells cultured at different densities (7,000 and 29,000 cells/cm²) in a 1% O₂ incubator for 4 days. Images were taken at 0, 3 and 4 days. Different replicates were used for each time point to prevent reoxygenation. Blue=nuclei stained with DAPI. Red=Dsred (HIF−). Green=GFP (HIF+). N=4 biological replicates per condition.

FIG. 9 shows that primary human hepatocytes experience anoxia during seeding in normoxia. Top: O₂ kinetics during the 1^{st} seeding step of hepatocytes into a Collagen I 24-well plate for 1 hour. Arrows indicate when plates were shaken to promote seeding. Bottom: O₂ kinetics during the 2^{nd} seeding step of hepatocytes for 4 hours. N=6-8 biological replicates.

FIG. 10 shows that primary human hepatocytes cultured in normoxia and physioxia exhibit a similar hypoxic transcriptomic response. Upregulated (left) and downregulated (right) genes in the Hallmark Hypoxia gene set for physioxic vs. uncultured (dark gray) and normoxic vs. uncultured (light gray) cultures. Genes with |log₂ (FC)|>1 for physioxic vs. uncultured are shown.

FIGS. 11A-11D show developing a reaction-diffusion model to predict pericellular O₂ tension in cell culture. FIG. 11A shows experimental (solid) and numerical (dashed) O₂ kinetics of media (EMEM+10% FBS+1% P/S) placed in a 5% O₂ incubator. FIG. 11B shows numerical (N) (dashed) and analytical (A) (dotted) O₂ kinetics for media in different culture vessel types placed in a 1% O₂ incubator. FIG. 11C shows reaction-diffusion model predictions of cells (20,000 cells/cm²) with different O₂ consumption rates (Vₘₐₓ) in a 6-well, 24-well, or 96-well plate in a 1% O₂ incubator. FIG. 11D shows surface area to height ratios for 6-well, 24-well, or 96-well plates given commonly used volumes for each culture vessel.

FIG. 12A-12B show controlling pericellular hypoxia and anoxia in MCF7 cultures. FIG. 12A-12B show Pericellular (solid) and gas phase (dashed) O₂ kinetics (left) and average O₂ tensions (right) for MCF7 cultures targeting pericellular hypoxia (1-2% O₂) (FIG. 12A) and anoxia (0-0.5% O₂) (FIG. 12B). Gas phase O₂ tension was manually changed to maintain pericellular hypoxia in (FIG. 12A). MCF7 cells were cultured in 6-well plates with 70,000 cells per well. N=4 biological replicates per condition.

FIGS. 13A-13C show proteomic characterization of the temporal differences between pericellular hypoxic and anoxic responses in 4T1. FIG. 13A shows principal component analysis (PCA) of protein abundances for 4T1 cells cultured in different O₂ tensions for 1, 2 or 3 days of culture. FIG. 13B shows volcano plots indicating significant downregulated or upregulated proteins (p_{adj}<0.05 and |log₂FC|≥0.5) for Hypoxia vs. Normoxia (top) or Anoxia vs. Normoxia (bottom) for 1 day, 2 days, or 3 days of culture. FIG. 13C shows PSEA using the Hallmark database, comparing different days in hypoxia (left) or anoxia (right). N=6-8 replicates per condition. NES=normalized enrichment score. N=3 biological replicates and N=2-3 technical replicates per condition.

### DETAILED DESCRIPTION

A cornerstone of biological research, cell culture aims to grow cells in conditions that simulate their native environment as closely as possible. Cell culture models serve as a tool for testing biological hypotheses before validating in vivo. Healthy and diseased tissues are isolated from patients and studied in vitro. In fact, cell culture techniques are used throughout the process of drug development to make “go/no-go” decisions and to manufacture adoptive cell therapies and regenerative medicines. Recent advances in this practice include scaffolds that mimic the extracellular matrix, self-assembly of pluripotent stem cells to form brain organoids, and patient-derived organoids that capture tumor heterogeneity in patients and predict therapeutic responses. Yet, one key discrepancy between in vitro and in vivo remain: the “normoxic” (i.e., room air) oxygen (O₂) tension in cell culture (141 mmHg) is dramatically higher than the O₂ tension of human tissues (3-100 mmHg) (1% O₂=7.7 mmHg O₂ at sea level).

Supraphysiological O₂ concentrations (hyperoxia) lead to excessive reactive oxygen species (ROS) production, resulting in cellular damage and dysregulated signaling. It is therefore not surprising that cells grown in physioxia experience less oxidative stress. Furthermore, hyperoxia degrades proteins containing a specific iron-sulfur cluster, disrupting diphthamide synthesis, purine metabolism, nucleotide excision repair, and electron transport chain (ETC) function. Many O₂-dependent enzymes require iron and copper metal cofactors, which are susceptible to oxidation in hyperoxia. Studies culturing cells in normoxia vs. physioxia have found aberrant T-cell activation, fibroblast senescence and mutation frequency, and chondrocyte differentiation in normoxia. However, the full impact of culturing cells in normoxia will remain unknown until physioxia becomes common practice.

The pericellular oxygen concentration (i.e., the oxygen concentration that cells actually experience within a cell culture vessel) is dramatically different than the gas phase in oxygen-controlling products. In fact, the pericellular oxygen tension is often lower than the surrounding gas phase due to cellular oxygen consumption. To create a system that accurately controls pericellular oxygen concentration for cell culture, we developed the product concept pericellular oxygen-controlling cell culture incubators. Unlike previous technologies, which control oxygen in the gas phase, these incubators flush nitrogen or air based on the pericellular oxygen concentration. These incubators use pericellular oxygen sensors as the feedback mechanism to control the flux of gas.

To address these concerns, tools to perform physiological cell culture have been developed and are commercially available. These products, including portable chambers, tri-gas incubators, and hypoxic workstations, consist of chambers that control O₂ in the atmosphere of cultured cells by adding compressed nitrogen. However, widespread adoption has been hampered by cost, laboratory space requirements, technical challenges, and rapid reoxygenation of cultures. Reoxygenation, which occurs when cell cultures are taken out of portable chambers or tri-gas incubators and exposed to normoxia, makes it difficult to recapitulate physiological O₂ tensions. Hypoxic workstations address this issue effectively. One major application for these products are hypoxic cell culture models, conducted at 0.5-1% O₂. Hypoxia occurs in both physiological (e.g., placenta, renal medulla, intestinal mucosa, germinal centers, bone marrow) and pathophysiological (e.g., infection, inflammation, solid tumors, ischemia) contexts; and is therefore an active area of research. Hypoxic cell culture was instrumental in the discovery of the prolyl hydroxylase (PHD)/hypoxia-inducible factor (HIF) axis, the mechanism by which cells sense and respond to low O₂. O₂-controlled cell culture is also performed to mimic physioxia, typically at 5% O₂.

In O₂-controlled cell culture, it is generally presumed that the atmospheric O₂ tension within incubators is equal to the pericellular O₂ tension, the O₂ concentration that cells experience. The pericellular O₂ tension is dependent on several rates: the O₂ diffusion within the cell culture media, O₂ transfer at the gas-media interface, and the cellular O₂ consumption. Gas-media O₂ transfer is the limiting rate. Culture vessel geometry, medium volume, and surface area also influence diffusion times. These parameters vary greatly based on user preference and experimental design, yet they are not reported. Experimental studies measuring pericellular O₂ tension indicate that confluent normoxic cultures can induce hypoxia. However, the impact of O₂ consumption rates in lower O₂ tensions is unclear, since consumption decreases as O₂ becomes limiting. We set out to assess how key cell culture parameters (i.e., cell type, cell density, medium volume, and culture vessel geometry) influence the relationship between atmospheric and pericellular O₂ tensions in O₂-controlled cell culture models. The theoretical nature of this relationship has been discussed in previous studies, but experimental data supporting it are lacking. After discovering that pericellular O₂ tension is vastly different from atmospheric O₂ tension, we explored how controlling pericellular O₂ tension could be used as a novel tool to study breast cancer cell responses in low O₂.

O₂-controlled cell culture, in which the O₂ concentration in an incubator's gas phase is controlled, is an indispensable tool to study the role of O₂ in vivo. For this technique, it is presumed that the incubator setpoint is equal to the O₂ tension that cells experience (i.e., pericellular O₂). Discovered herein is that physioxic (5% O₂) and hypoxic (1% O₂) setpoints regularly induce anoxic (0% O₂) pericellular tensions in both adherent and suspension cell cultures. Electron transport chain inhibition ablates this effect, indicating that cellular O₂ consumption is the driving factor. RNA-seq revealed that primary human hepatocytes cultured in physioxia experience ischemia-reperfusion injury due to anoxic exposure followed by rapid reoxygenation. To better understand the relationship between incubator gas phase and pericellular O₂ tensions, developed herein is a reaction-diffusion model that predicts pericellular O₂ tension a priori. This model revealed that the effect of cellular O₂ consumption is greatest in smaller volume culture vessels (e.g., 96-well plate). By controlling pericellular O₂ tension in cell culture, discovered herein is that MCF7 cells have stronger glycolytic and glutamine metabolism responses in anoxia vs. hypoxia. MCF7 also expressed higher levels of HIF2A, CD73, NDUFA4L2, etc. and lower levels of HIFIA, (A9, VEGFA, etc. in response to hypoxia vs. anoxia. Proteomics revealed that 4T1 cells had an upregulated epithelial-to-mesenchymal transition (EMT) response and downregulated reactive oxygen species (ROS) management, glycolysis, and fatty acid metabolism pathways in hypoxia vs. anoxia. Collectively, these results reveal that breast cancer cells respond non-monotonically to low O₂, suggesting that anoxic cell culture is not suitable to model hypoxia. We demonstrate that controlling atmospheric O₂ tension in cell culture incubators is insufficient to control O₂ in cell culture and introduce the concept of pericellular O₂-controlled cell culture.

Despite the widespread use of O₂-controlling chambers in hypoxia-related research, studies quantifying pericellular O₂ concentrations and their impact on cellular response are surprisingly lacking. In the current study, we discovered vast differences between incubator setpoints and pericellular O₂ tensions in every cell type tested.

Our results highlight a major challenge with portable chambers and tri-gas incubators: cultures cannot be conditioned to start at the desired O₂ tension due to rapid reoxygenation of media upon exposure to normoxia. Without media conditioning, MCF7 cultures can take 1-14 hours to reach 1% O₂ depending on experimental set-up. Not only does this time difference introduce significant variability between experiments, but it also suggests that shorter hypoxic experiments may not even reach hypoxia. We also show that changing cell culture parameters induce a sustained difference of HIF stabilization kinetics for at least 5 days for two different cell lines. Unless a hypoxic workstation with preconditioned media is used, pericellular O₂ tension must be determined to report accurate O₂-controlled incubation times.

Challenging the belief that incubators accurately control O₂ for cell cultures, we discovered that pericellular anoxic tensions are common in both physioxic (5% O₂) and hypoxic (1% O₂) conditions due to cellular O₂ consumption. This effect occurred in both primary human adherent and suspension cultures, with commonly used cell densities, medium volumes, and culture vessel types. Furthermore, our results suggest that physioxic cultures are routinely hypoxic. O₂ tension in physioxia can vary greatly (0.1-4.5% O₂) depending on experimental set-up, engendering reproducibility concerns. These findings are a major concern for physioxic culture of stem cell expansion and differentiation, and controlling pericellular O₂ tension may improve our understanding of these processes.

Our pericellular O₂ tension results for primary human hepatocyte cultures, a prominent model in drug metabolism, underpin how the incubator setpoint is a poor indicator of the O₂ cells experience. Hepatocytes cultured in physioxia experienced anoxia for 24 hours, followed by a rapid reoxygenation upon media exchange. RNA-seq results indicate that O₂ fluctuations in these conditions induce an upregulation in cellular responses to mitochondrial and NADPH oxidase ROS production (e.g., superoxide, hydrogen peroxide). Oxidative stress increased cell death via upregulation of TNF (apoptosis) and IL-1β (pyroptosis) production. Damage-associated molecular patterns (DAMPs) released by dying cells activate toll-like receptor (TLR) and pattern recognition receptor (PRR) pathways, inducing key signatures of a sterile inflammatory response: complement activation, inflammasome complex assembly, MHC class II upregulation, and IL-6 production. During reoxygenation, hepatocytes increased mitochondria and ribosome biogenesis to meet ATP and protein translation demands as the cells recovered from hypoxic exposure. Ultimately, these results indicate that physioxic culture of hepatocytes drives a cellular response mimicking liver ischemia-reperfusion injury, a major risk factor in graft dysfunction in liver transplantation.

We developed a reaction-diffusion model, which accurately predicted pericellular O₂ tensions of MDA-MB-231 cultures at various incubator setpoints. This novel tool can design O₂-controlled cell culture experiments, modulating parameters like the cell density, culture vessel type, or medium volume to achieve desired pericellular O₂ concentrations. Our finding that the effect of cellular O₂ consumption increases as culture vessel size decreases suggests that smaller vessels (e.g., 96-well plates) should be avoided for O₂-controlled cell culture. This observation carries significant implications for immune cell culture, which are typically done at high cell densities in small culture vessels. Cell growth rates may be considered as a function of O₂ tension. O₂ consumption rates (Vₘₐₓ) for different cell types may be needed. Such studies may uncover O₂ consumption trends applicable to most cell types.

Using the reaction-diffusion model and manipulation of gas phase O₂, we performed the first investigation into the relationship between pericellular O₂ tension and biological response. We investigated the metabolic, transcriptomic, and translational responses to hypoxia (1-2% O₂) and anoxia (0% O₂) in two different breast cancer cell lines. Quantification of 14 genes associated with hypoxia (11 are direct HIF targets) and HIFIA/HIF2A suggest distinct transcriptional responses in hypoxia and anoxia. In anoxia, we found higher transcription in HIFIA, hypoxic markers (VEGFA, CA9), metabolism (LDHA, PDK1, SLCIA5), mitophagy (BNIP3, BNIP3L), and CD274 (PD-L1) compared to hypoxia. Conversely, HIF2A, ATF4, NDUFA4L2, and NT5E (CD73) was upregulated in hypoxia. The increase in HIFIA transcription in anoxia may occur through ROS-induced PI3 kinase (PI3K) and protein kinase C (PKC) pathways, since our proteomic analysis suggests that ROS production is higher in anoxia.

A previous study reported that maximum HIF1 DNA-binding activity occurs at pericellular 0.5% O₂ and sharply decreases as tension approaches 0% O₂. This suggests that in anoxic culture, HIF is maximally stabilized as cultures approach anoxia (rather than in anoxia per se) inducing a strong HRE transcriptional response. Ultimately, 0.5% pericellular O₂ may be the ideal setpoint for hypoxic cell culture, as long as tensions do not drop to anoxic levels. The post-translational modification of both HIF1 and HIF2, as well as downstream responses, under various low O₂ tensions, ranging from 0% to 3% O₂ may be considered. These studies aim to provide a mechanistic understanding of how cells respond differently to these tensions.

To further understand low O₂ tension responses, we performed an in-depth characterization of the 4T1 proteome in response to low O₂ tensions. This analysis suggests that the global translational response to anoxia is stronger and faster than the hypoxic response. Yet, the responses are distinct: we found an upregulation of EMT proteins in hypoxia and an increased ROS response in anoxia for 72 hours of culture. EMT is a critical step in hypoxia-driven metastasis and metastatic breast cancer represents the most advanced stage of the disease. Our findings suggest that mitochondrial dysfunction and ROS production are higher in anoxia than in hypoxia.

A hypoxic workstation or conditioned media was not used indicating that our O₂-controlled cultures did not immediately reach the desired O₂ tension. Responses in different O₂ tensions were not differentiated whether they were due to HIF-dependent or HIF-independent mechanisms, and a mechanistic understanding of the differences between hypoxic and anoxic responses was not provided. Cancer cell responses to low O₂ tensions were examined after culturing them in supraphysiological O₂ (i.e., normoxia), which could potentially influence the responses.

Ultimately, our exploration of breast cancer responses to low O₂ tensions suggest that anoxia is not suitable to model hypoxia. This is fortified by the fact that the median O₂ tension in breast tumors is 10 mmHg (1.3% O₂). Equally important, our findings uncover that breast cancer cells respond non-monotonically to low O₂, since many aspects of the low O₂ response are upregulated in hypoxia compared to anoxia.

O₂ is a critical factor for mammalian bioenergetic homeostasis and serves as a substrate for over 200 enzymatic reactions. O₂-controlled cell culture attempts to mimic O₂ tensions that cells are exposed to in vivo and is therefore a critical tool for biological research. Herein, we report the discovery that the metric used to determine O₂ concentration for in vitro cultures, the incubator setpoint, is a poor indicator of the O₂ tension cells actually experience (i.e., pericellular O₂ tension) due to cellular O₂ consumption. Standard physioxic (2% to 5% O₂, preferably 5% O₂) and hypoxic (0.5% to 1% O₂, preferably 1% O₂) protocols routinely induce anoxia (0% to 0.5% O₂, preferably 0% O₂). Furthermore, in physioxic culture, pericellular O₂ tension is highly dependent on cell culture parameters, making reproducibility difficult. Highlighting the significance of these findings, we demonstrated that a key drug metabolism model, primary human hepatocytes, undergo an effect similar to ischemia-reperfusion injury when cultured in physioxia. To address these challenges, we developed a reaction-diffusion model that predicts pericellular O₂ tension a priori. Using this tool, we controlled pericellular O₂ tension in two breast cancer models to explore transcriptional and translational responses to hypoxia and anoxia. We discovered that breast cancer cells respond non-monotonically to low O₂. Overall, this work calls for a fundamental change to how O₂-controlled cell culture is performed and suggests that pericellular O₂-controlled cell culture is necessary to accurately model O₂ tension.

In one aspect, described herein are methods of culturing cells comprising. The methods may comprise incubating the cells in a bioreactor with an inflow of gas. Numerous embodiments are further provided that can be applied to any aspect of the present invention described herein. For example, in some embodiments, the cells may be contained in a cell culture container. The methods may further comprise monitoring the pericellular oxygen concentration. The methods may further comprise adjusting the pericellular oxygen concentration to a target concentration by adjusting the inflow of gas.

In one aspect, described herein are bioreactors comprising a process for incubating cells contained in a cell culture container, wherein the bioreactor comprises an inflow of gas; a process for monitoring the pericellular oxygen concentration; and a process for adjusting the pericellular oxygen concentration to a target concentration by adjusting the inflow of gas. The methods may comprise incubating the cells in a bioreactor with an inflow of gas. Numerous embodiments are further provided that can be applied to any aspect of the present invention described herein. For example, in some embodiments, the cell culture container comprises an oxygen sensor to measure the pericellular oxygen concentration.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

As used herein, the term “the pericellular oxygen concentration” refers to the oxygen concentration that cells actually experience within a cell culture vessel. The pericellular oxygen concentration is dramatically different than the gas phase in oxygen-controlling products. In fact, the pericellular oxygen tension is often lower than the surrounding gas phase due to cellular oxygen consumption.

As used herein, the term “a cell culture gas” refers to a specific gas composition required for a stable and sterile environment. Preferred conditions for a cell culture comprise a stable and sterile environment with a specific temperature, humidity, and gas composition. In some embodiments, the cell culture gas is nitrogen, carbon dioxide, oxygen, and/or ambient air. For example, carbon dioxide is essential for regulating the pH of the culture media, as it acts as a buffer to maintain the pH within the physiological range for the cells to grow. To attain the desired experimental concentrations, O₂ (solute gas) is mixed with other gases (balance gases). Nitrogen (N₂) is commonly used alone for this purpose, but some researchers use also argon (Ar). These two gases (N₂ and Ar) are inert (i.e. do not undergo chemical reactions under experimental conditions). Another common solute gas for cell culture is carbon dioxide (CO₂). It interacts with the bicarbonate buffer in the cell culture medium, stabilizing the pH at about the optimum level (˜7.4).

### Incubators/Bioreactors

The disclosure describes an incubator that is capable of maintaining control over the pericellular oxygen tension in cell cultures. A final product set-up would look like a standard cell culture incubator with a humidity, CO₂, temperature and oxygen sensors. The incubator has inlets with mass flow controllers for CO₂ and N₂. The incubator has an outlet that allows ambient air into the incubator. The incubator will require that cell culture vessels with associated contactless sensors that measure oxygen are used to culture cells. Commercial examples of this include Presens SDR and Pyroscience OXSP5. The incubator has LED and photodiode meters that can be attached to cell culture vessels. The incubator registers the oxygen sensors' readouts and display to the end user. The incubator allows the end user to set the pericellular oxygen concentration (determined by contactless oxygen sensor). The incubator adjusts the oxygen concentration of the surrounding gas phase (measured by incubator oxygen sensor) until the desired oxygen is reached. Surrounding gas phase is lowered by introducing N₂ and increased by allowing ambient air to enter. All of this is accomplished through PID control. The incubator has multiple chambers to control oxygen concentration to different levels. “Incubator” includes tri-gas incubators and workstations.

The results show proof-of-concept of this strategy, using a Thermo Fisher Scientific 3130 incubator and Pyroscience OXSP5 sensors manually attached to the bottom of cell culture vessels. However, this approach is not feasible for typical end user use. The novel aspect of this disclosure is that convention assumes the pericellular oxygen concentration is equal to the gas phase oxygen concentration. In other words, current companies and end users assume there is no need to measure and/or control pericellular oxygen concentration.

In one aspect, described herein are culture incubators. The culture incubators may comprise a cell culture container; and an inflow of gas, wherein cells are contained in the cell culture container. The culture incubators may further comprise an oxygen sensor to monitor the pericellular oxygen concentration of the cells in the cell culture container. In some embodiments, the pericellular oxygen concentration is adjusted to a target oxygen concentration by adjusting the inflow of a cell culture gas.

In some embodiments, the oxygen sensor is located at the bottom of the cell culture container. In some embodiments, the pericellular oxygen concentration is measured using a mathematically modeled equation that predict pericellular oxygen tension in an oxygen-controlled cell culture. In some embodiments, the mathematically modeled equation is Michaelis-Menten kinetics. In some embodiments, the cell culture gas is nitrogen, argon, carbon dioxide, oxygen, and/or ambient air. In some embodiments, the culture incubator further comprises a humidity sensor, a carbon dioxide sensor, a temperature sensor, and/or an oxygen sensor. In some embodiments, the culture incubator further comprises an inlet with a mass flow controller for carbon dioxide and/or a mass flow controller for nitrogen.

In some embodiments, the culture incubator comprises an outlet that allows ambient air into the incubator. In some embodiments, the cells are eukaryotic cells or prokaryotic cells. In some embodiments, the cells are eukaryotic cells; and the eukaryotic cells are cell strains derived from an animal, a plant, or an insect, a primary culture product, or a fungus. In some embodiments, the cells are prokaryotic cells; and the prokaryotic cells are bacteria including Escherichia coli, Bacillus subtilis, cyanobacteria, Actinomycetes methanogen, extreme halophile, or hyperthermophile. In some embodiments, the target oxygen concentration is for anoxic conditions.

In some embodiments, the target oxygen concentration is 0% to about 0.5% oxygen. In some embodiments, the target oxygen concentration is 0% oxygen, about 0.1% oxygen, about 0.2% oxygen, about 0.3% oxygen, about 0.4% oxygen, or about 0.5% oxygen. In some embodiments, the target oxygen concentration is for hypoxic conditions. In some embodiments, the target oxygen concentration is about 0.5% to about 1% oxygen. In some embodiments, the target oxygen concentration is about 0.5% oxygen, about 0.6% oxygen, about 0.7% oxygen, about 0.8% oxygen, about 0.9% oxygen, or about 1% oxygen. In some embodiments, the target oxygen concentration is for physioxic conditions. In some embodiments, the target oxygen concentration is about 2% to about 5% oxygen. In some embodiments, the target oxygen concentration is about 2% oxygen, about 2.5% oxygen, about 3% oxygen, about 3.5% oxygen, about 4% oxygen, about 4.5% oxygen, or about 5% oxygen. In some embodiments, the culture incubator is a bioreactor. In some embodiments, the cell culture container is a 6-well plate, 12-well plate, a 24-well plate, a 96-well plate, a T25 flask, a T75 flask, a T175flask, or a T225 flask.

### EXAMPLES

The invention now being generally described, it will be more readily understood by reference to the following examples, which are included merely for purposes of illustration of certain aspects and embodiments of the present invention, and are not intended to limit the invention.

### Example 1: Methods

### In Vitro O₂ Measurements

Adhesive optical O₂ sensor spots (OXSP5-ADH-STER, PyroScience GmbH) were used to measure the O₂ concentration of media and cell cultures as previously described. Sensors were placed on the culture vessel surface and a cable adapter (SPADBAS, PyroScience GmbH) was glued on the opposite side of the culture vessel (lined up with the sensor). Glue was allowed to dry overnight. Optical fiber cables (SPFIB-BARE, PyroScience GmbH) were placed within the adapters and connected to a computer via a meter (FireSting O₂, PyroScience GmbH). The 100% O₂ calibration was performed with aerated Dulbecco's phosphate-buffered saline (DPBS), and the 0% O₂ calibration was performed using the factory calibration value. For cell culture experiments, cells were seeded in sensor-containing culture vessels and pericellular O₂ was measured. A temperature probe (TDIP15, PyroScience GmbH) connected to the meter was placed inside the same incubator as the sensor-containing culture vessels. To measure the O₂ concentration at the top of the media or cell culture wells, needle-like probes (OXROB10, PyroScience GmbH) were attached to a micromanipulator (MM33, PyroScience GmbH) and placed at the media-gas interface, such that the probes were submerged at the top layer of the media. Holes were drilled in plate lids to allow the probes to reach the media.

### Cell Culture

Mycoplasma-free cell lines, MDA-MB-231 (ATCC HTB-26), MCF7 (ATCC HTB-22), 4T1 (ATCC CRL-2539), and primary mammary epithelial (ATCC PCS-600-010) were obtained from the American Type Culture Collection (ATCC). MDA-MB-231, MCF7, and 4T1 were maintained in Leibovitz's L-15 medium (Cytiva), Eagles' Minimum Essential Medium (EMEM) with L-glutamine (Quality Biological) and Dulbecco's Minimum Essential Medium (DMEM) (Corning), respectively, with 10% fetal bovine serum (FBS, Corning) and 1% penicillin/streptomycin (P/S, Invitrogen). Mammary epithelial cells were cultured in basal medium (ATC PCS-600-030) with cell growth kit (ATCC PCS-600-040). MCF7 and MDA-MB-231 hypoxia-inducible factor (HIF) reporter cell lines were transduced and selected as previously described. For all cultures, passage number did not exceed 20.

Human dendritic cells (DCs) were differentiated from cryopreserved CD14+ monocytes over a period of 7 days as previously described (Posch W, et al. J Vis Exp. Dec. 24 2016; (118)). In brief, monocytes were seeded into a 6-well plate in ImmunoCult™-ACF Dendritic Cell Medium (Stemcell Technologies), supplemented with recombinant human granulocyte-macrophage colony-stimulating factor (GM-CSF) (50 ng/mL) (R&D Systems) and recombinant human interleukin-4 (IL-4) (50 ng/mL) (R&D systems).

Primary human hepatocytes (HUCPG, Lonza) were cultured following the manufacturer's protocol. Briefly, hepatocytes were thawed in thawing medium (MCHT50, Lonza) and then seeded onto a 24-well plate (BioCoat Collagen I, Corning) using plating medium (MP100 and MP250, Lonza). The seeding process involved gentle shaking every 15 minutes for 1 hour (Seed 1), followed by replacement of the plating medium and another 4 hours of incubation (Seed 2). Subsequently, the hepatocytes were then cultured (T=0 hour) using maintenance medium (CC-3198, Lonza), which was exchanged after 24 hours. Finally, cells were harvested for RNA-seq after 36 hours of culture.

For O₂-controlled experiments, cells were incubated within a tri-gas incubator (Heracell VIOS 160i, Thermo Fisher Scientific), which was kept closed throughout the duration of the experiment. Cells were not removed from O₂-controlled environments for passaging. Cultures that were taken out of the tri-gas incubator for processing were immediately placed on ice and lysed.

### Cellular Hypoxia Kinetics

MCF7 cells were seeded onto 60 mm glass dishes (Cellvis) overnight and incubated with Hoechst 33342 (NucBlue Live ReadyProbes Reagent, Invitrogen), 10 μM Image-iT™ Red Hypoxia Reagent (Invitrogen), and 1 μM Celltracker Orange for 30 min at 37° C. Cells were placed within a humidified incubator at 37° C., 5% CO₂, and 1% O₂/99% N₂ (O₂ Module S, CO₂ Module S, and Temp Module S, Zeiss) attached to a confocal microscope (LSM 880 with Airyscan, Zeiss). Images were taken every 30 min for 12 hours. Average fluorescence per cell was calculated using Fiji. Briefly, cell area multiplied by background fluorescence was subtracted from the cell's integrated density. At least thirty cells were analyzed per image.

### Hypoxia-Inducible Factor (HIF) Stabilization Kinetics

For the cell density experiments, MCF7 HIF reporter cells were used as previously described (Godet I, et al. Nature Communications. 2019 Oct. 24 2019; 10 (1): 4862). Briefly, these cells contain two vectors: Vector 1 consists of a Cre gene modified by the addition of an O₂-dependent degradation domain, which is transcriptionally controlled by a HIF-DNA binding sequence (HRE). Vector 2 consists of a red fluorescent protein gene (Dsred) with a stop codon flanked by tandem loxP sites, followed by a green fluorescent protein gene (GFP). MCF7 reporter cells were seeded into a 12-well plate at different densities (7,000 and 29,000 cells/cm²) overnight. Cells were cultured at 1% O₂ for 4 days, incubated with Hoechst 33342 and confocal images were taken daily. Percentage of GFP+ among MCF7 reporter cells was determined using Fiji (Threshold and Analyze Particles). For culture vessel type experiments, MDA-MB-231 HIF reporter cells were seeded onto a 24-well plate or T25 flask (30,000 cells/cm²) and cultured at 1% 02 for 5 days. Flow cytometry (Cytoflex S, Beckman Coulter) of live singlets was used to determine the GFP positive fraction.

### Mitochondrial Inhibition

MCF7 were seeded overnight in a 24-well plate and incubated at 5% O₂ for 24 hours. 10 μL of DPBS or sodium azide (Sigma Aldrich) (final concentration=5 mM) were added into cultures. For cytotoxicity studies, MCF7 were seeded overnight and incubated with ±5 mM sodium azide for 6 hours at 5% O₂. Cells were incubated with 1:1000 live/dead dye (LIVE/DEAD Fixable Kit, Thermo Fisher Scientific) for 1 hour. Flow cytometry (Attune NxT, Thermo Fisher Scientific) of live singlets was used to determine the cell viability.

### Cell Viability Time Course

MCF7 were seeded into a T25 flask (20,000 cells/cm²) overnight and cultured at 18.6%, 5%, or 1% O₂ for 6 days. Every 2 days, media was harvested, and trypan blue staining and a hemocytometer were used to determine live and dead detached cells. Attached cells were trypsinized (Trypsin-EDTA, Gibco) and counted using the same method.

Library Preparation with polyA Selection and Illumina Sequencing

RNA was extracted immediately from hepatocytes (NucleoSpin RNA, Macherey-Nagel) and quantified using a Qubit 2.0 Fluorometer (Life Technologies). Cells were removed from O₂-controlled incubators, immediately placed on ice, and then lysed using Lysis Buffer RA1. RNA integrity was checked using Agilent TapeStation 4200 (Agilent Technologies). RNA sequencing libraries were prepared using the NEBNext Ultra II RNA Library Prep for Illumina per the manufacturer's protocol (New England Biolabs). Briefly, mRNAs were enriched with Oligod (T) beads. Enriched mRNAs were fragmented for 15 min at 94° C. First strand and second strand cDNA were subsequently synthesized. cDNA fragments were end repaired and adenylated at 3′ ends, and universal adapters were ligated to cDNA fragments, followed by index addition and library enrichment by PCR with limited cycles. The sequencing libraries were validated on the Agilent TapeStation 4200 and quantified by using Qubit 2.0 Fluorometer as well as by quantitative PCR (KAPA Biosystems). The sequencing libraries were multiplexed and clustered onto a flowcell. After clustering, the flowcell was loaded onto the Illumina instrument (HiSeq 400 or equivalent) according to the manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq were converted into FASTQ files and de-multiplexed using Illumina bcl2fastq 2.20 software. One mismatch was allowed for index sequence identification. FASTQ files were trimmed with Trimmomatic and the quality was analyzed with FastQC. The human genome (GRCh38.p14) was annotated and reads were aligned using STAR. Gene counts were determined using FeatureCounts. Differential gene expression analysis was performed using DESeq2. Gene set enrichment analysis (GSEA) was performed using the clusterProfiler package in R.

### Reaction-Diffusion Model

The unsteady state diffusion equation (1) with initial and boundary conditions (2-4) were used to describe O₂ transfer between cell culture media and gas phase, where C is the concentration of O₂, D is the diffusivity coefficient, and k_{L}a is the mass transfer coefficient. x=0 is the bottom of the well and x=L is the media height. A diffusivity coefficient of 0.09684 cm²/hr was used and experimental diffusion data were used to determine k_{L} values. An analytical solution was determined (5-6). Michaelis-Menten kinetics were used for the reaction-diffusion model (7), where Vₘₐₓ is the maximum O₂ consumption rate and Kₘ is the O₂ concentration at which the reaction rate is half of Vₘₐₓ. Numerical values were determined using the MATLAB PDE solver (MathWorks).

$$\frac{dC}{dt} = D \frac{{\partial }^{2} C}{\partial {t}^{2}} \qquad ( 1 )$$

$$C ( x , t = 0 ) = {C}_{0} \qquad ( 2 )$$

$$\frac{dC ( x = 0 , t )}{dx} = 0 \qquad ( 3 )$$

$$- D \frac{dC ( x = L , t )}{dx} = {k}_{L} a ( C - {C}_{gas} ) \qquad ( 4 )$$

$$Θ ( ζ , τ ) = {\sum }_{m = 1}^{∞} \frac{2 sin ( {λ}_{m} ) {e}^{- {λ}_{m}^{2} τ} cos ( {λ}_{m} ζ )}{cos ( {λ}_{m} ) sin ( {λ}_{m} ) + {λ}_{m}} \qquad ( 5 )$$

$$Θ = \frac{C - {C}_{gas}}{{C}_{0} - {C}_{gas}} ζ = \frac{x}{L} τ = \frac{Dt}{{L}^{2}} \qquad ( 6 )$$

$$\frac{dC}{dt} = D \frac{{\partial }^{2} C}{\partial {t}^{2}} - \frac{{V}_{max} C}{{K}_{M} + C} \qquad ( 7 )$$

### Extracellular Metabolite Quantification

Media was removed from cell cultures and centrifuged at 250 g for 5 min. Resulting supernatant was stored at −20° C. and used for metabolite quantification. Live cells from cultures were determined using trypan blue staining and a hemocytometer. Glucose uptake and lactate secretion were quantified by an Agilent 1260 high performance liquid chromatography (HPLC) Infinity II System equipped with a BioRad Aminex HPX-87H ion exchange column (300 mm×7.8 mm) operated at 60° C. with a refractive index detector (RID) operated at 50° C. The mobile phase was 14 mM sulfuric acid with a flow rate of 0.6 mL/min. The injection volume of each sample was 10 μL. Peak areas for each compound for concentrations ranging from 0.125 g/L to 5 g/L were used to make calibration curves in OpenLab ChemStation (LTS 01.11) and then used to calculate metabolite quantifications. Glutamate and glutamine concentration was determined using the Glutamine/Glutamate-Glo Assay (Promega). Metabolite concentration was normalized by the cell number at each time point.

### Reverse Transcription and Quantitative PCR (RT-qPCR)

RT-qPCR was performed as previously described. RNA was extracted from MCF7 cultures (NucleoSpin RNA, Macherey-Nagel). Cells were removed from O₂-controlled incubators, immediately placed on ice, and then lysed using Lysis Buffer RA1. RNA quality was checked using a NanoDrop One spectrophotometer (Thermo Fisher Scientific). Reverse transcription was conducted using a High-Capacity cDNA Reverse Transcription Kit (Applied Biosystems) on a MyCycler thermal cycler (Bio-Rad). Gene expression was quantified using the following TaqMan Gene Expression Assays (Thermo Fisher Scientific) on an MX3005P QPCR System (Agilent Technologies): VEGF-A (Hs00900055_m1), (A9 (Hs00154208_m1), LDHA (Hs01378790_g1), PDK1 (Hs01561847_m1), NT5E (Hs00159686_m1), PRKAA2 (Hs00178903_m1), BNIP3L (Hs00188949_m1), BNIP3 (Hs00969291_m1), HIFIA (Hs00153153_m1), HIF2A (Hs00909569_g1), SLC2A1 (Hs00892681_m1), SLCIA5 (Hs01056542_m1), SLC7A11 (Hs00921938_m1), NDUFA412 (Hs00220041_m1), BNIP3 (Hs00969291_m1), BNIP3L (Hs00188949_m1), NT5E (Hs00159686_m1), (D) 274 (Hs00204257_m1), and ACTB (Hs01060665_g1).

### Bulk Proteomic Sample Preparation

Cells were removed from O₂-controlled incubators, immediately placed on ice, centrifuged at 4° C., resuspended in Mass Spectrometry grade Water (Fisher Scientific, W6500), and then frozen at −80° C. Cells were lysed by heating at 90° C. for 10 min, protein concentrations for each lysate were measured using a Nanodrop (A205). Proteins were digested to peptides per the SCoPE2 protocol. Briefly, 10 μg of protein per sample were digested in a solution containing 100 mM triethylammonium bicarbonate at pH 8.5 (TEAB) (Sigma Aldrich, T7408), benzonase nuclease (Millipore Sigma, Cat E1014) and Trypsin Gold (Promega, V5280). The protease was added at a 1:20 enzyme to substrate ratio and LC-MS grade water was added to maintain its concentration at 20 ng/μL. The reaction was carried out for 12 hours at 37° C.

Digested peptides were subsequently dried down in a SpeedVac vacuum evaporator and resuspended in 200 mM TEAB (pH 8.5). Samples were randomized and labelled using either do, d4 or d8 of mTRAQ mass tags (SciEx, 4440015, 4427698, and 4427700) in a reaction that maintained ⅓^{rd} organic phase and the manufacturer's suggested reagent to peptide ratio (1U for 100 μg of peptides). The labelling was carried out for 2 hours at room temperature and excess, unreacted label was quenched by adding hydroxylamine (Sigma Aldrich, 467804) to 0.2% v/v and leaving at room temperature for 1 hour. Two samples from each label were randomly selected and 50 nng analyzed in data-dependent acquisition (DDA) mode to evaluate labelling efficiency.

Samples from each label were combined in equal amounts to make a plexDIA set that was dried down and resuspended in 0.1% formic acid (Thermo Fisher, 85178) in MS grade water to a final concentration of 1 μg/μL. Samples within a plexDIA set were randomly paired; a few samples across labels were repeated across multiple sets.

### Proteomics Data Acquisition

The separation was performed at a constant flow rate of 200 nL/min using a Dionex UltiMate 3000 UHPLC, and 1 μL of sample was loaded onto a 25 cm×75 μM IonOpticks Odyssey Series column (ODY3-25075C18). The separation gradient was 4% buffer B (80% acetonitrile in 0.1% Formic Acid) for 11.5 min, a 30 second ramp up to 12% B followed by a 63 min linear gradient up to 32% B. Subsequently, buffer B was ramped up to 95% over 2 min and maintained as such for 3 additional min. Finally, buffer B was dropped to 4% in 0.1 min and maintained for 19.9 additional min.

The mass spectra were analyzed using a Thermo Scientific Q-Exactive mass spectrometer from min 20 to 95 of the LC method. An electrospray voltage of 1700V was applied at the liquid-liquid junction of the analytical column and transfer line. The temperature of the ion transfer tube was 250° C., and the S-lens RF level was set to 30.

Bulk data was collected in Data Independent Acquisition (DIA) mode, the duty cycle consisted of a total of 3 MS1 scans and 30 MS2 scans. All MS1 scans were conducted at 140,000 resolving power with a maximum injection time of 300 milliseconds and a target AGC of 3e6 with a scan range covering 378-1290 m/z. All MS2 scans were conducted at 35,000 resolving power, a maximum inject time of 110 ms, AGC target of 3e6 and normalized collision energy of 27. MS2 scans had variable isolation widths: 10 MS2 scans of 17 m/z isolation width (isolation window) followed the first and second MS1 scan respectively, the third MS1 was followed by 5 windows of 33 m/z, 2 windows of 40 m/z, 1 window of 80 m/z and a final window of 120 m/z.

### Proteomics Data Processing

DIA-NN (version 1.8.1) was used to search the raw files from each run. A predicted spectral library was made using the swissprot mouse FASTA database and in silico labelled to have mTRAQ as a fixed mod (+140.0949630177) on each trypsin digested peptide.

Peak height was used for quantification with a scan window of 1, mass accuracy of 10 ppm and MS1 accuracy of 5 ppm, MBR was enabled, and search outputs were filtered at 1% Q value. The following commands were employed by use of the additional commands dialogue: -fixed-mod mTRAQ 140.0949630177, nK, -channels mTRAQ, 0, nK, 0:0; mTRAQ, 4, nK, 4.0070994:4.0070994; mTRAQ, 8, nK, 8.0141988132:8.0141988132}, -peak-translation, -ms1-isotope-quant, -ms1-base-profile, -ms1-subtract 2.

The report file containing filtered peptide level output was processed using R. First, the peptide level data was collapsed/summarized to a run x protein matrix using the diannmaxlfq function from the ‘diann’ R package. Subsequently, the matrix was log₂ transformed and the protein levels in each run were normalized for differential loading amounts by adding to each protein value, the median of the difference between the vector of protein levels for that run to the vector of median values across all runs. Relative protein levels were obtained by subtracting away the mean value across runs for each protein. In order to correct for biases specific to each mTRAQ label, kNN imputation (k=3) was performed and ComBat was used with mTRAQ labels as batch covariates. Post batch correction two matrices were used for further analysis, one with imputed values and the other where the imputed values had been set back to NA. Differential protein expression analysis was performed using limma. Protein set enrichment analysis (PSEA) was performed using the clusterProfiler package in R.

### Statistical Analysis

All data were presented as the mean±standard error of the mean (SEM). Biological replicate indicates a unique culture for a given condition. Statistical analyses were performed using Prism 9 software (GraphPad). Number of replicates and statistical tests used are outlined in the figure captions. Values represent the mean±standard error of the mean. Significance levels are reported as *p<0.05, ** p<0.01, *** p<0.001, **** p<0.0001.

### Example 2: 1% O₂ Media Conditioning can Take Over 5 Days and is Reoxygenated within Minutes

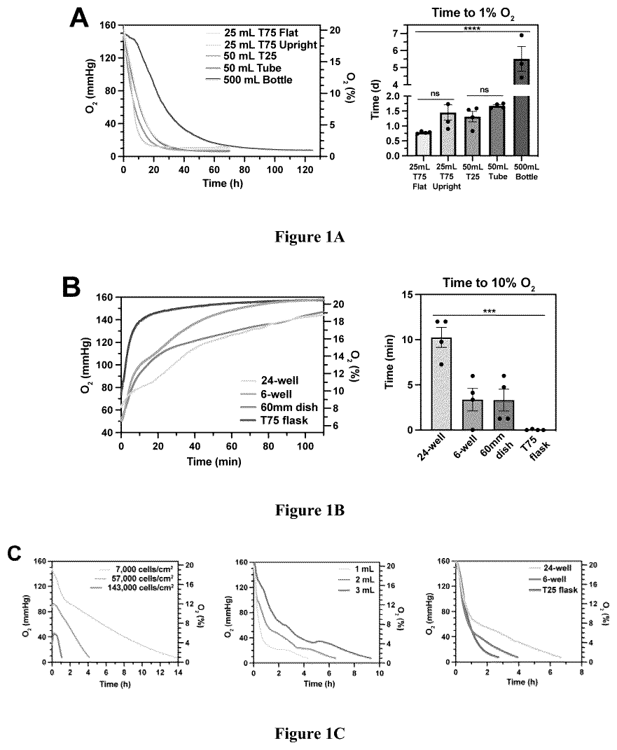

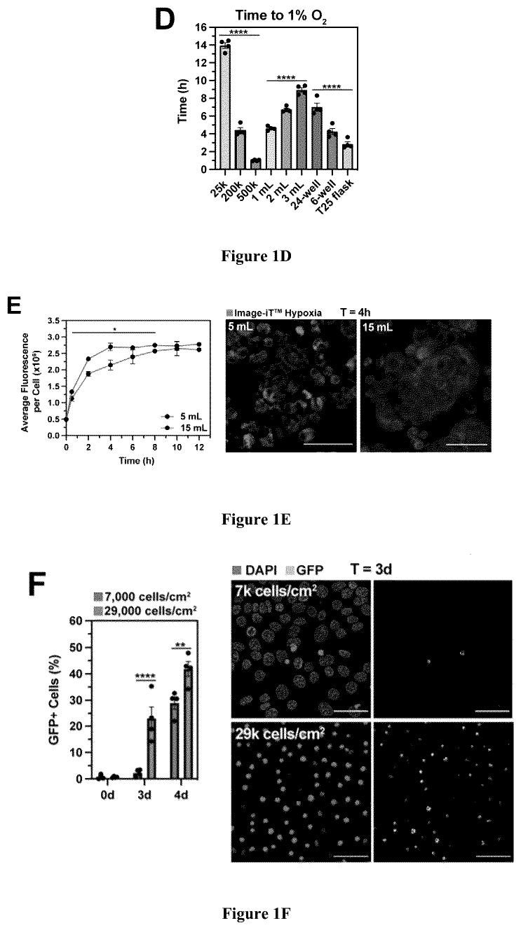

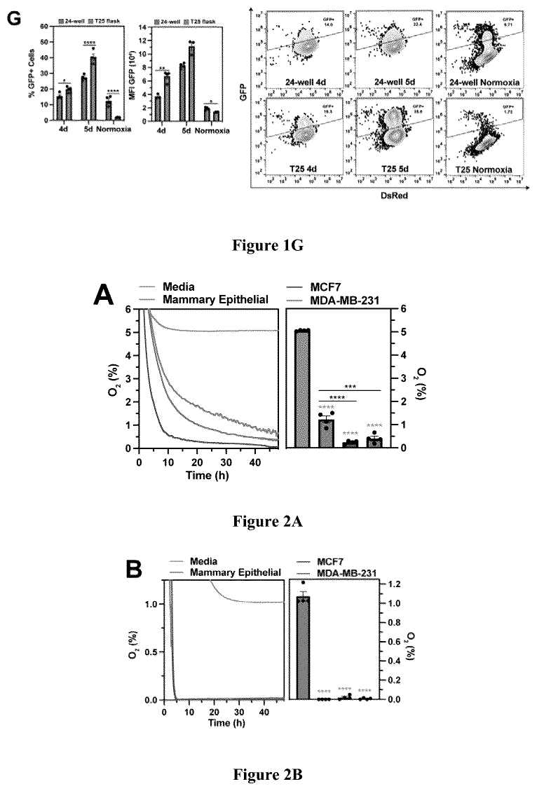

For O₂-controlled cell culture experiments, media is typically conditioned to the desired O₂ tension and added to the cells at the start of the experiment. This procedure ensures that cells experience the desired O₂ tension immediately. We investigated how long it would take to condition 25-500 mL of media for hypoxic (1% (2) experiments, since conditioning times are not reported. The required time was far longer than anticipated: over 1 day for 25 mL (upright T75 flask) and over 5 days for 500 mL (FIG. 1A). The type of culture vessel or tube used did not change conditioning time for 25 or 50 mL of media. To understand media reoxygenation kinetics, 500 mL of 1% O₂ medium was removed from a tri-gas incubator and aliquoted into different culture vessels containing O₂ sensors. By the time the media reached the culture vessels, the O₂ concentration was >6% O₂ (FIG. 1B). Depending on the surface area of the medium in different culture vessels, the medium reached 10% O₂ within seconds to 10 min. These results indicate that 1% O₂ media conditioning requires surprisingly long periods-more than 5 days for large volumes. Furthermore, 1% O₂ media is rapidly reoxygenated when removed from controlled O₂ atmospheric environments, indicating that portable chambers and tri-gas incubators are not suitable to condition media.

### Example 3: Cell Density, Medium Volume, and Culture Vessel Type Influence HIF Stabilization Kinetics

We investigated how cell density, medium volume, and culture vessel type influence the time it takes normoxic cultures to reach 1% O₂. Non-invasive optical sensor spots were used to measure pericellular O₂ tension. For MCF7 breast cancer cultures, all three parameters influenced the time to 1% O₂, ranging from 1-14 hours (FIG. 1C-D). Cell density had the largest effect, suggesting that cellular O₂ consumption plays a key role in the induction of hypoxia in vitro.

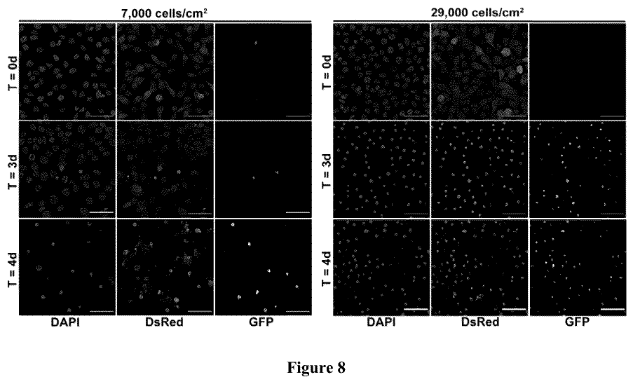

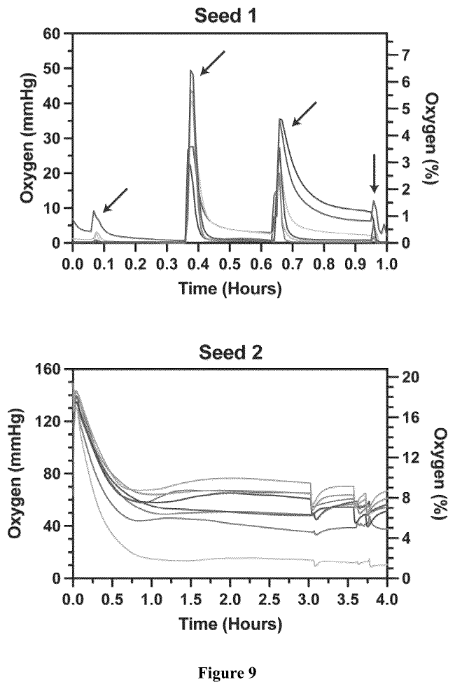

Next, we explored whether medium volume influenced cellular hypoxia kinetics. MCF7 cultures in 60 mm dishes containing either 5 mL or 15 mL of media were placed inside a 1% O₂ incubator. Cellular hypoxia was evaluated using a hypoxia-responsive fluorescent dye (Image-iT™ Hypoxia) for 12 hours. As expected, the cells in the 5 mL condition reached a maximum cellular fluorescence sooner than the 15 mL cultures: 4 vs. 10 hours (FIG. 1E, FIG. 7).

To understand if cell density or culture vessel type influenced HIF stabilization in 1% O₂ culture, MCF7 and MDA-MB-231 HIF reporter cell lines were cultured at two cell densities for 4 days and the percentage of GFP-positive cells was determined using fluorescent microscopy. After 3 days, 2.2% of cells plated at the lower cell density were GFP-positive whereas 22.9% of cells plated at a higher density were GFP-positive (FIG. 1F, FIG. 8). We then tested whether different culture vessel types would induce a similar effect for MDA-MB-231 reporter cells. Consistent with our findings in FIG. 1C, T25 flask cultures had a higher percentage of GFP-positive cells after 4 and 5 days compared to 24-well cultures (FIG. 1G). Collectively, we have demonstrated that cell density, medium volume, and culture vessel type, parameters that vary between experiments and are not reported, greatly influence cellular hypoxia and HIF stabilization kinetics. These results suggest that consistent HIF stabilization kinetics in 1% O₂ culture can only be obtained by using a workstation and conditioned media.

### Example 4: Cellular O₂ Consumption can Induce Anoxia in Both 5% and 1% O₂ Culture

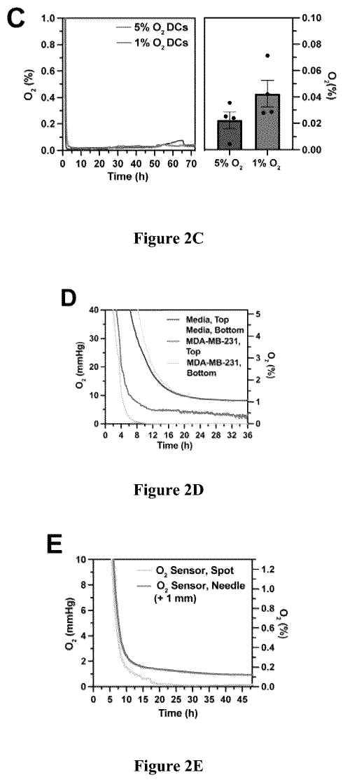

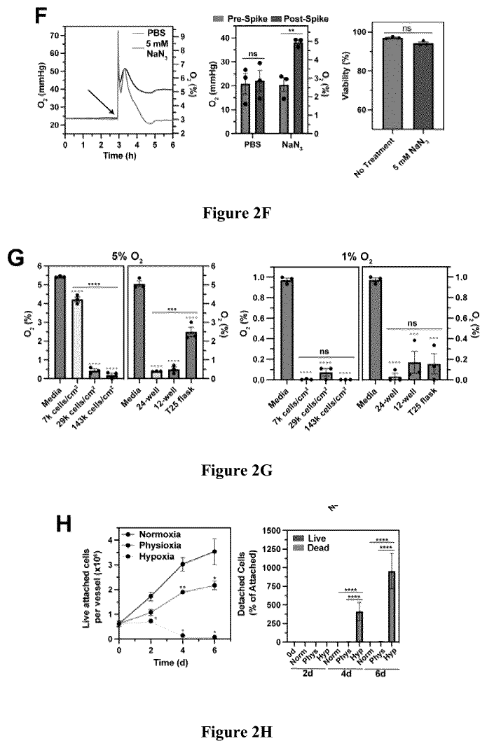

After discovering that cellular O₂ consumption drives the induction of hypoxia in 1% O₂ incubators, we hypothesized that it would also influence the pericellular O₂ tension. To test this, sub-confluent (21×10³ cells/cm²) MCF7, MDA-MB-231, and primary human mammary epithelial cells were cultured in physioxic (5% O₂) and hypoxic (1% (2) conditions for 72 hours and the pericellular O₂ concentration was measured. Cell-free media O₂ tensions matched the incubator setpoints, indicating that the O₂ sensor spots were accurately recording and the incubator O₂ sensors were calibrated (FIG. 2A-2B). In physioxia, the pericellular O₂ was strikingly low-below 1.5% O₂ for all three lines, and MCF7 cultures were anoxic (anoxia defined as <0.5% O₂). In hypoxia, the pericellular O₂ of all cultures became anoxic (0% O₂) within 5 hours. Next, we investigated whether this effect also occurred in suspension culture. Activated human dendritic cells (DCs) were cultured in physioxia or hypoxia for 72 hours. Both cultures were immediately and sustainably anoxic (0% O₂) (FIG. 2C).

If cellular O₂ consumption did indeed affect pericellular O₂ tension, there would be an axial O₂ gradient in these cultures. To test this hypothesis, O₂ tension at the media-gas interface of MDA-MB-231 cultured at 1% O₂ was measured using needle O₂ microsensors (top) and pericellular O₂ tensions were measured using sensor spots (bottom). FIG. 2D illustrates that in these cultures, there is a gradient of 0.4-0% O₂ from the top to the bottom of the well after 36 hours. In wells without cells, both the top and bottom of the well reached 1% O₂ as expected. To validate O₂ sensor spot readings, needle microsensors were placed 1 mm above sensor spots in 1% O₂ MDA-MB-231 cultures using a micromanipulator. Sensor spots recorded 0% O₂ and needles recorded 0.1% O₂, indicating that the two probes were in good agreement (FIG. 2E).

To further validate the role of cellular O₂ consumption in pericellular O₂ tension, we tested whether inhibiting oxidative phosphorylation would ablate axial gradients. Upon addition of sodium azide (NaN₃, complex IV inhibitor), the pericellular O₂ tension of MCF7 cultures rapidly rose from 3% O₂ to the incubator setpoint of 5% O₂, whereas PBS (vehicle) spiked cultures returned to 3% O₂ with continued incubation (FIG. 2F, left panel). To confirm that this effect was not due to NaN₃ cytotoxicity, we confirmed that there was no significant change in MCF7 cell viability when incubated with NaN₃ at 5% O₂ for 6 hours (FIG. 2F, right panel).

We next explored how cell density and culture vessel type influenced the gradient between atmospheric and pericellular O₂ tensions. In physioxic MCF7 cultures, cell densities of 7,000, 29,000, and 143,000 cells/cm² in a 12-well plate induced pericellular O₂ tensions of 4.2%, 0.5%, and 0.1% O₂, respectively. Different culture vessels (24-well plate, 12-well plate, and T25 flask) also influenced MCF7 tensions, ranging from 0.4-2.5% O₂ (FIG. 2G, left panel). In hypoxia, neither cell density nor culture vessel type change pericellular tensions in MCF7 cultures: all were anoxic (<0.2% O₂) (FIG. 2G, right panel).

After determining that all hypoxic MCF7 cultures tested were anoxic, we sought to understand how pericellular anoxia affected cell viability. MCF7 cells were cultured in normoxia, physioxia, or hypoxia for 6 days and cell proliferation and viability were evaluated. Cells cultured in normoxia and physioxia proliferated throughout the 6-day period (FIG. 2H, left panel). However, cells cultured in hypoxia did not proliferate, and the majority were dead and detached after 4 days (FIG. 2H, right panel).

Collectively, these experiments show that cellular O₂ consumption drives pericellular O₂ far below the incubator setpoint, inducing anoxia in both physioxic and hypoxic MCF7, MDA-MBA-231, and human DC cultures. Furthermore, in physioxic MCF7 culture, pericellular O₂ tension is highly dependent on cell culture parameters, ranging from 0.1-4.2% O₂.

### Example 5: Setting the Incubator to Physioxia Mimics Ischemia-Reperfusion Injury in Hepatocyte Culture

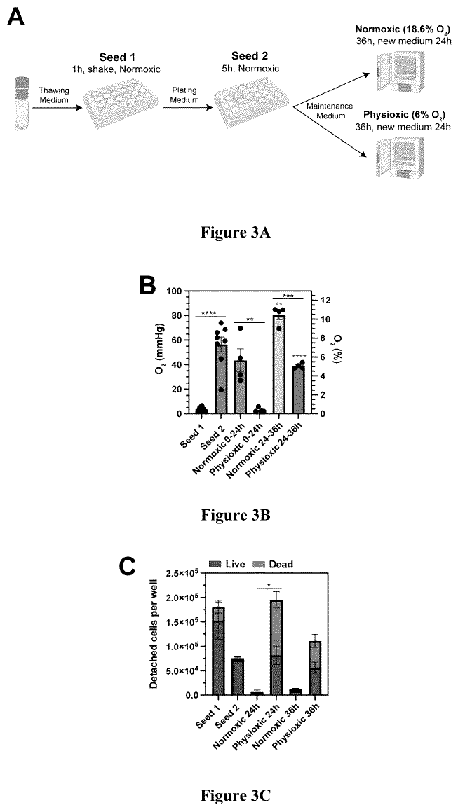

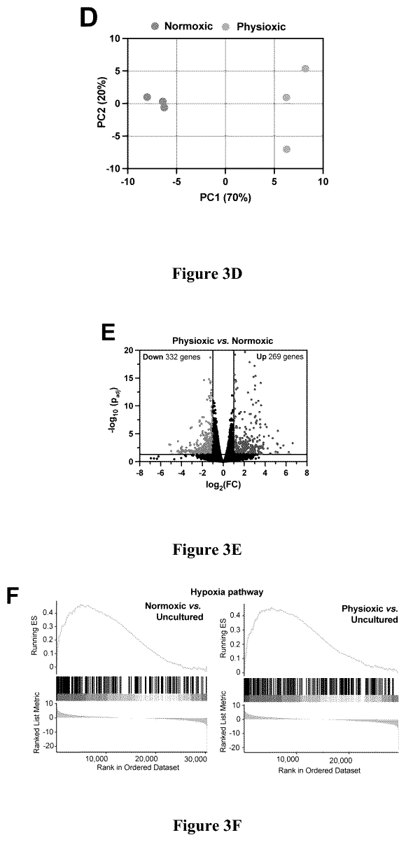

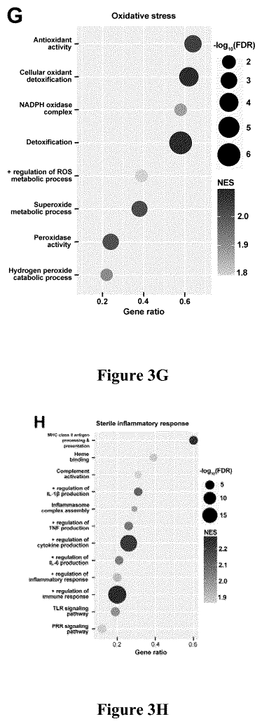

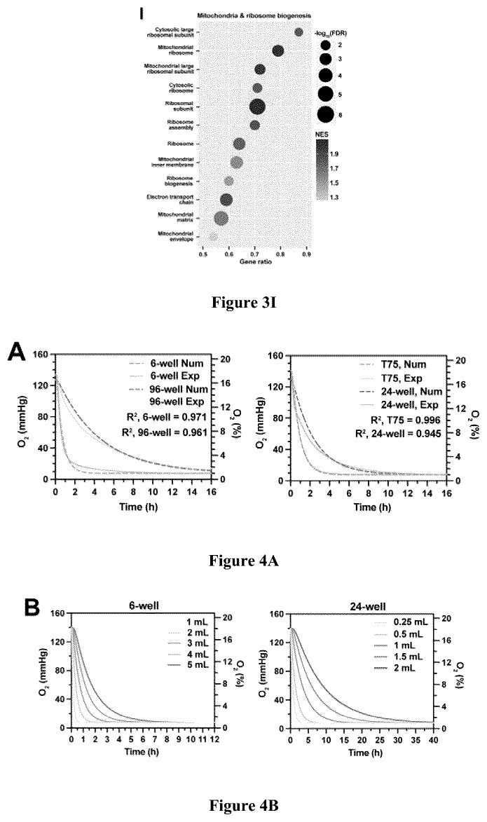

We next explored how the difference between the incubator setpoint and pericellular O₂ tension can impact the physiological relevance of cell culture models. Because of their high O₂ consumption rate and widespread use as an in vitro drug metabolism model, primary human hepatocytes were used for these studies. Hepatocytes were seeded and cultured in either normoxic (18.6% O₂) or physioxic (6% O₂) conditions for 36 hours (FIG. 3A).

During the first seeding step, which was conducted in normoxia, hepatocytes were anoxic (0.5% (2) (FIGS. 3B and 9). During the first 24 hours of culture, hepatocytes cultured in normoxia were physioxic (5.6% O₂) and hepatocytes cultured in physioxia were anoxic (0.4% O₂). Unexpectedly, when media was exchanged after 24 hours, both types of cultures underwent reoxygenation for the duration of the experiment. FIG. 3C illustrates that the pericellular anoxia experienced by hepatocytes cultured in physioxia increased the number of detached cells after 24 hours, the majority of which were dead.

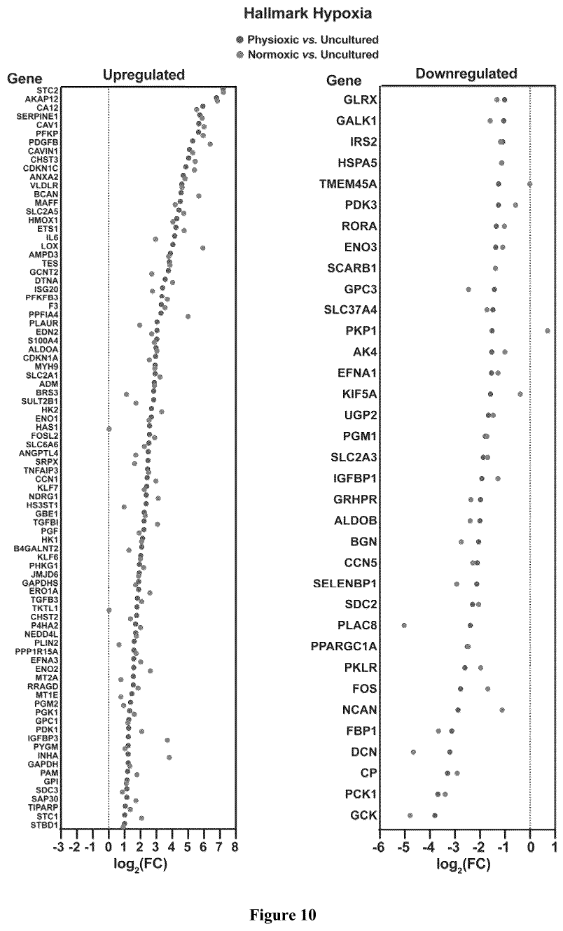

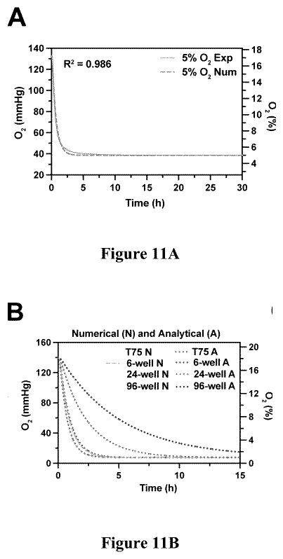

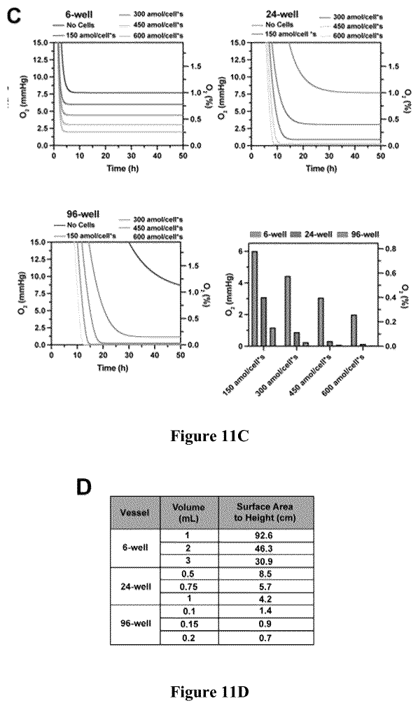

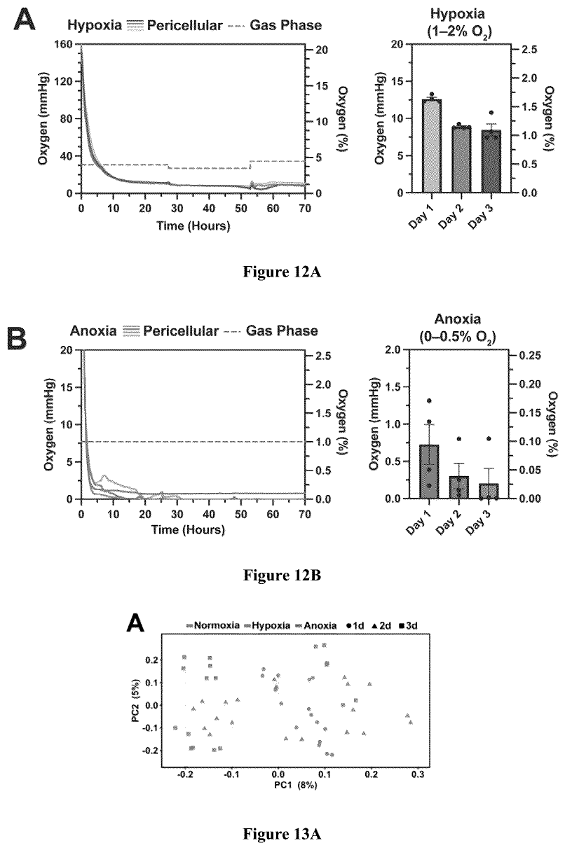

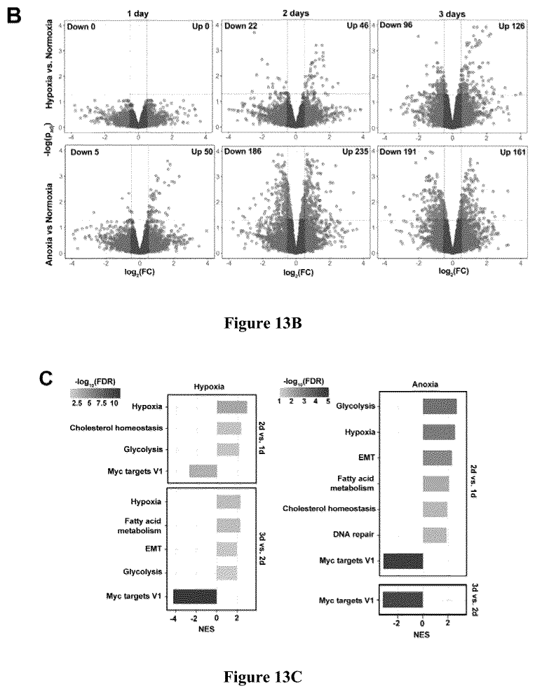

To investigate how the pericellular O₂ tension influenced hepatocyte physiology, we assessed gene expression by RNA-seq of uncultured hepatocytes, and hepatocytes cultured in normoxia or physioxia after 36 hours. Principal component analysis (PCA) of the transcriptome shows clustering of replicates by O₂ tension, as expected (FIG. 3D). Differential gene expression analysis (physioxic vs. normoxic) found 269 upregulated and 332 downregulated genes (FIG. 3E). Gene set enrichment analysis (GSEA) revealed an upregulation in hypoxia-associated genes in normoxic and physioxic cultured cells compared to uncultured hepatocytes (FIG. 3F), indicating that the 1-hour anoxic seeding step induced a hypoxic response. Examining 195 genes within the Hallmark Hypoxia gene set reveals that the hypoxic response was similar between physioxic and normoxic cultures (FIG. 10). In addition, GSEA suggests that hepatocytes cultured in physioxia mounted an oxidative stress response (FIG. 3G). Corroborating the higher cell death and detachment, physioxic cultured hepatocytes also had an upregulation in IL-1β production, TNF production, TLR signaling, and PRR signaling pathways, indicating a sterile inflammatory response (FIG. 3H). Lastly, hepatocytes in physioxia had upregulated mitochondrial and ribosomal biogenesis pathways (FIG. 3I). Taken together, these results indicate that setting the incubator to physiological O₂ conditions mimics ischemia-reperfusion injury in hepatocytes due to their cellular O₂ consumption.

### Example 6: Developing a Reaction-Diffusion Model to Predict Pericellular O₂ Tension in Cell Cultures

Measuring pericellular O₂ tension for every O₂-controlled cell culture experiment would be cumbersome and expensive. We hypothesized that a computational model could predict pericellular tension a priori, given cell density, O₂ consumption rate, culture vessel type and medium volume. Such a tool would reduce the need for experimental measurements.

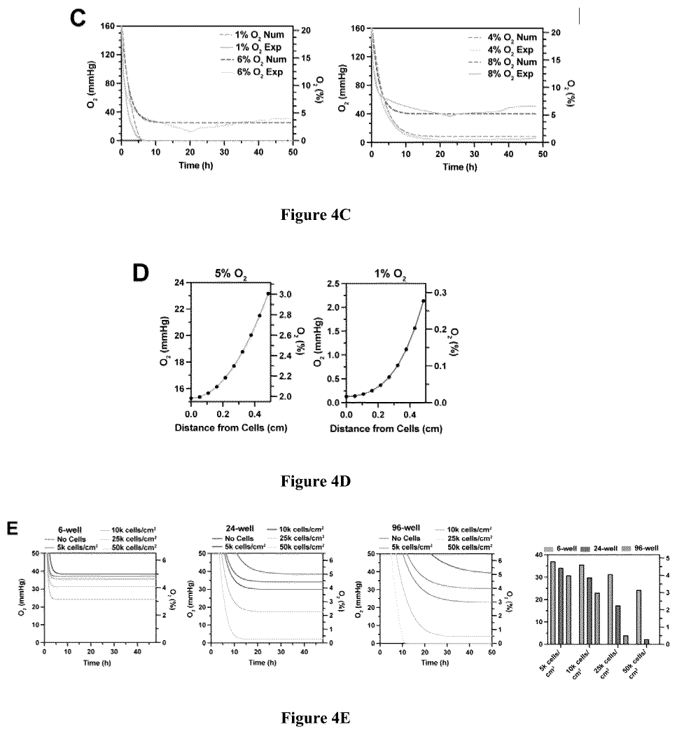

We first examined whether the unsteady state diffusion equation could describe O₂ transfer kinetics between cell culture medium and incubator gas phases. Coefficient of determination (R²) values suggested that experimental and numerical values that describe the O₂ transfer between normoxic media and 1% O₂ gas phase in different culture vessels were in good agreement (FIG. 4A). The diffusion model also predicted equilibration of normoxic media in a 5% O₂ incubator (FIG. 9A). In addition, the diffusion model's analytical solution was also comparable to numerical solutions for different culture vessel types (FIG. 9B).

After validating the diffusion model, we applied it to investigate the dependency of O₂ transfer kinetics on medium volume in a 24-well plate and 6-well plate. FIG. 4B shows that although commonly used medium volumes for 24-well and 6-well plates modestly impact the time to 1% O₂, there is a substantial difference in kinetics between 24-well and 6-well plate wells. 6-well plate volumes of media require 0.5-12 hours to reach 1% 02, whereas 24-well plate requires 2.5-40 hours. Importantly, the rate of equilibration for the same medium volumes (1 mL and 2 mL) is substantially lower in 24-well plate than 6-well plate wells. These findings indicate that the gas phase-pericellular O₂ differential increases as culture vessel surface area decreases (FIG. 2G) because of a decrease in O₂ transfer rates.

Next, we developed a reaction-diffusion model to describe pericellular O₂ tension in cell cultures within O₂-controlled environments. Michaelis-Menten kinetics were used to model cellular O₂ consumption. This model predicts pericellular O₂ tension values for MDA-MB-231 cultured at 1%, 4%, 6%, and 8% O₂ with reasonable Michael-Menten parameters (Vₘₐₓ=450 amol cell⁻¹ sec⁻¹ and Kₘ=1 μM)+3 (FIG. 4C). For further validation, we tested whether the model predicted axial O₂ gradients like those experimentally determined in FIG. 2D. The model predicts gradients of 2.0-3.0% O₂ and 0-0.3% O₂ for 30,000 cells/cm² MDA-MB-231 cultures in physioxia and hypoxia, respectively (FIG. 4D), in good agreement with experimental data.

Using the reaction-diffusion model, we next examined the influence of cell density on pericellular O₂ tension in different culture vessel types in physioxia. The model predicts that the highest cell density will have a modest influence on pericellular tension in 6-well plate cultures (3.2% (2), but it will induce anoxia in 24-well (0.4% O₂) and 96-well plate cultures (0% O₂) (FIG. 4E). Lastly, we investigated the influence of cellular O₂ consumption (Vₘₐₓ) in hypoxia (FIG. 9C). Both cell density and O₂ consumption predictions suggest that the smaller the culture vessel surface area, the greater the impact of cellular O₂ consumption on pericellular O₂ tension. This effect is due to the decreasing media surface area to height as culture vessel size decreases (FIG. 10D). Taken together, we establish that a reaction-diffusion model can predict pericellular O₂ tension in O₂-controlled cell culture. Furthermore, using the model, we discovered that the effect of cellular consumption on pericellular O₂ tension is highly dependent on the culture vessel type, increasing as culture vessel size decreases.

### Example 7: Pericellular Anoxia Induces Stronger Metabolic Reprogramming Compared to Pericellular Hypoxia in MCF7 Cells

The studies presented thus far demonstrate that standard hypoxic cell culture (1% O₂) routinely induces anoxia due to cellular O₂ consumption. Because anoxia is not physiologically relevant in vivo, we asked whether anoxia is suitable to model hypoxia. To explore this concept, we controlled pericellular O₂ tension to investigate cancer cell responses to pericellular hypoxia (1-2% O₂) vs. pericellular anoxia (0-0.5% O₂).

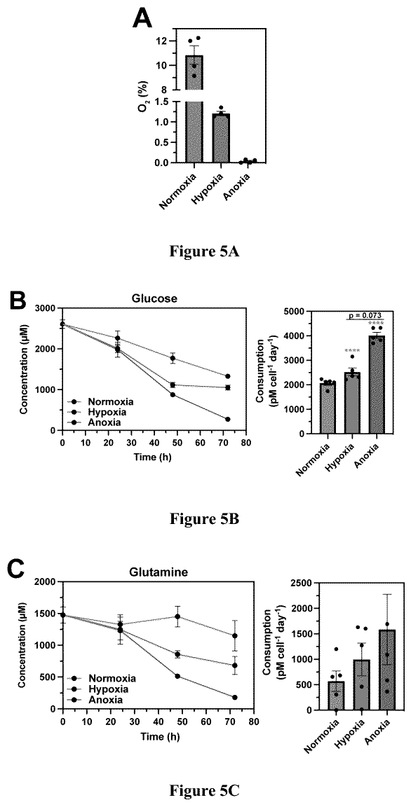

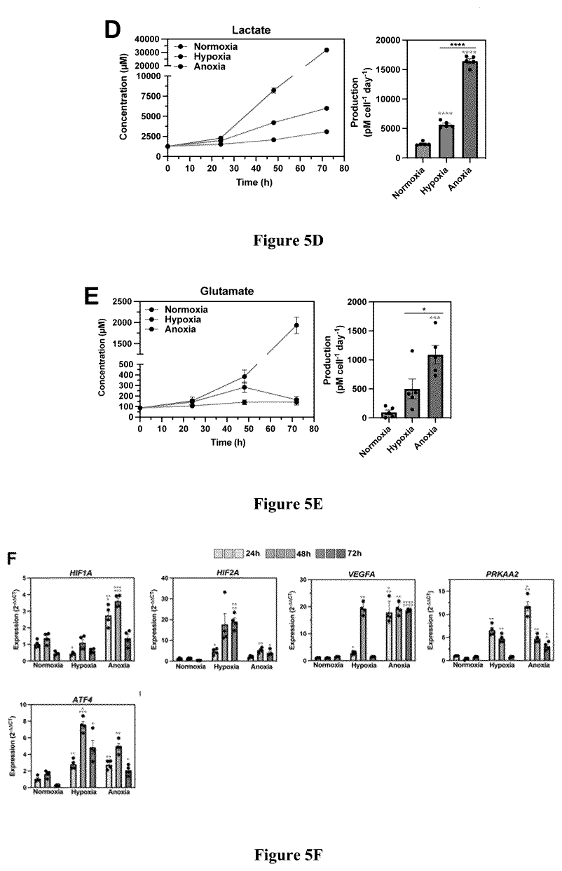

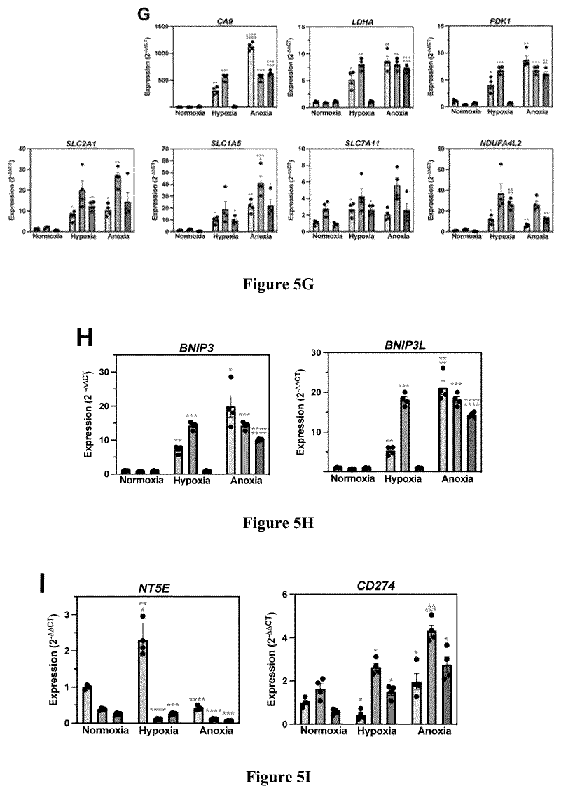

First, we examined MCF7 metabolic reprogramming in response to different pericellular O₂ tensions. Expected metabolic changes in response to hypoxia include an increase in (i) glucose consumption due to increased uptake and glycolytic flux, (ii) extracellular lactate from decreased TCA cycle flux and increased lactate transport, (iii) glutamine uptake to replenish TCA cycle intermediates for lipid metabolism, and (iv) extracellular glutamate secretion, which promotes cancer cell proliferation. MCF7 cells were cultured for 72 hours in 18.6%, 3.5-4.5%, and 1% O₂ incubators, resulting in supraphysiologic (10.8% O₂), hypoxic (1.2% O₂), and anoxic (0% (2) pericellular tensions, respectively (FIG. 5A). To maintain pericellular hypoxia, the incubator's gas phase was adjusted from 4% to 3.5% O₂ at 27 hours and from 3.5% to 4.5% O₂ at 53 hours (FIG. 10A). These changes in gas phase O₂ tension led to a rapid change in pericellular O₂ tension. Additionally, a constant 1% O₂ gas phase was sustained to maintain pericellular anoxia throughout the entire time course (FIG. 10B). Consumption (glucose and glutamine) and production (lactate and glutamate) rates trended higher with decreasing pericellular O₂ tension over the 72-hour time course (FIGS. 5B-5E). For example, lactate and glutamate production was 2.2- and 1.6-fold higher for cells in anoxia compared to hypoxia, respectively. Only the anoxic MCF7 cultures exhibited increased rates after 24 hours, whereas hypoxic and normoxic cultures maintained constant rates. These findings suggest that for MCF7 cells, anoxia induces a stronger metabolic reprogramming response than hypoxia does.

### Example 8: Pericellular Hypoxia Vs. Anoxia Induces Distinct Transcriptional HIFα and HRE Gene Responses in MCF7 Cells

We examined MCF7 transcriptional responses to low O₂ tensions at 24, 48, and 72 hours, including genes associated with (i) the hypoxic response (e.g., hypoxia inducible factor 1 subunit alpha (HIFIA), hypoxia inducible factor 2 subunit alpha (HIF2A), vascular endothelial growth factor A (VEGFA), protein kinase AMP-activated catalytic subunit alpha 2 (PRKAA2), and activating transcription factor 4 (ATF4)); (ii) metabolic reprogramming (e.g., carbonic anhydrase 9 (CA9), lactate dehydrogenase A (LDHA), pyruvate dehydrogenase kinase 1 (PDK1), solute carrier family 2 member 1 (SLC2A1, GLUT1), solute carrier family 1 member 5 (SL (1A5), solute carrier family 7 member 11 (SLC7A11, xCT), and NADH dehydrogenase 1 alpha subcomplex, 4-like 2 (NDUFA41.2)); (iii) mitophagy (e.g., Bcl-2 interacting protein 3 (BNIP3) and Bcl-2 interacting protein 3 like (BNIP3L)); and (iv) the immunosuppressive tumor microenvironment (TME) (e.g., cluster of differentiation 274 ((D) 274, PD-L1) and 5′-nucleotidase ecto (NT5E, CD73) (FIG. 5F-5I). Nearly all of these genes are direct HIF targets (i.e., hypoxia-responsive element (HRE)-controlled genes), excluding PRKAA2, ATF4, and SL (1A5.

In anoxia, VEGFA, CA9, PDK1, BNIP3, and BNIP3L show elevated expression compared to normoxia throughout the time course. However, in hypoxia, these genes steadily increased expression and peaked after 48 hours, followed by a drop to normoxic expression levels after 72 hours. Interestingly, HIFA expression was different in hypoxia and anoxia: HIFIA had higher expression in anoxia (6- and 3-fold after 24 and 48 hours, respectively) and HIF2A had higher expression in hypoxia (5-fold after 72 hours) (FIG. 5F). In addition, MCF7 had higher expression of PRKAA2 (AMPK α2 subunit) in anoxia (2- and 4-fold after 24 and 72 hours, respectively), suggesting lower ATP availability in anoxia. Interestingly, hypoxia induced 2-fold higher expression of ATF4 after 48 hours than did anoxia, indicating a stronger integrated stress response in hypoxia. Glycolytic genes had higher expression in anoxia (LDHA 7-fold and PDK1 9-fold after 72 hours). SL (2A1 (GLUT1) expression levels also trended higher in anoxia throughout the time course. SL (1A5 expression levels were 2-fold higher in anoxia after 48 hours. Overall, these results support the metabolic profiles in FIG. 5B-5E.

BNIP3 and BNIP3L expression levels were higher in anoxia after 72 hours, suggesting an upregulation in mitophagy in anoxia. Lastly, in the context of immunosuppression, hypoxia induced 6-fold higher expression of NT5E (CD73, extracellular AMP to adenosine conversion) after 24 hours. Anoxia induces 2-fold higher expression of (D) 274 (PD-L1) after 48 hours. Overall, these results indicate that hypoxia and anoxia induce distinct expression profiles in both HIFA and HRE responsive genes in MCF7.

### Example 9: Proteomic Characterization of the Temporal Differences Between Pericellular Hypoxic and Anoxic Responses in 4T1 Cells

After looking at transcriptional responses to hypoxia and anoxia, we aimed to better understand changes in protein expression in response to low O₂ tensions. To this end, we applied plexDIA to understand how the proteome changes in response to hypoxia and anoxia in a murine triple negative breast cancer (TNBC) cell line (4T1). PCA of the proteome shows clustering by O₂ tension and by day (FIG. 10A). Furthermore, hypoxic and anoxic samples shift away from normoxic samples along PC1 over time, indicating continuing changes during low O₂ responses.

For the hypoxic response, differential protein abundance analysis indicates no significantly upregulated or downregulated proteins after 1 day of culture, with the maximum response occurring after 3 days. On the other hand, the anoxic response had 50 upregulated and 5 downregulated proteins after day 1, and the response peaked after only 2 days. The number of changing proteins was higher in anoxia than hypoxia for all three days (FIG. 10B).

Protein set enrichment analysis (PSEA) was performed to compare each low O₂ response between days. In agreement with the differential protein abundance analysis, PSEA suggests that the anoxic response is faster and peaks by day 2: most of the changes occur for 2 days vs. 1 day (FIG. 10C). For both O₂ conditions, hypoxia-associated pathways are upregulated throughout the time course, including hypoxia, glycolysis, cholesterol homeostasis, fatty acid metabolism, and epithelial-to-mesenchymal transition (EMT). Interestingly, Myc targets were downregulated in both tensions for all days, which may contribute to the reduction in proliferation at low O₂ tensions. Overall, this temporal characterization of the proteome suggests that the response to anoxia is stronger and faster than the response to hypoxia in 4T1 cells.

### Example 10: Characterizing Proteomic Differences in Metabolic Reprogramming Between Pericellular Hypoxic and Anoxic Responses in 4T1 Cells

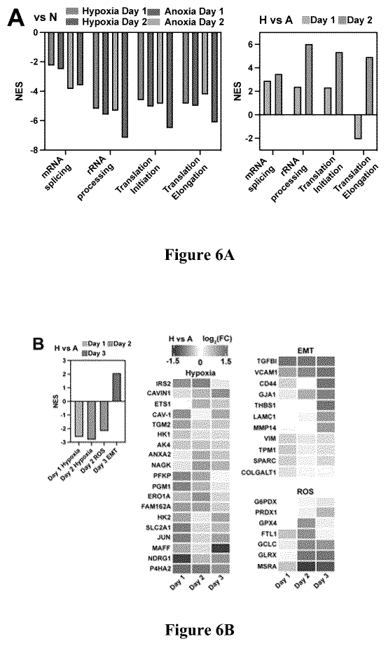

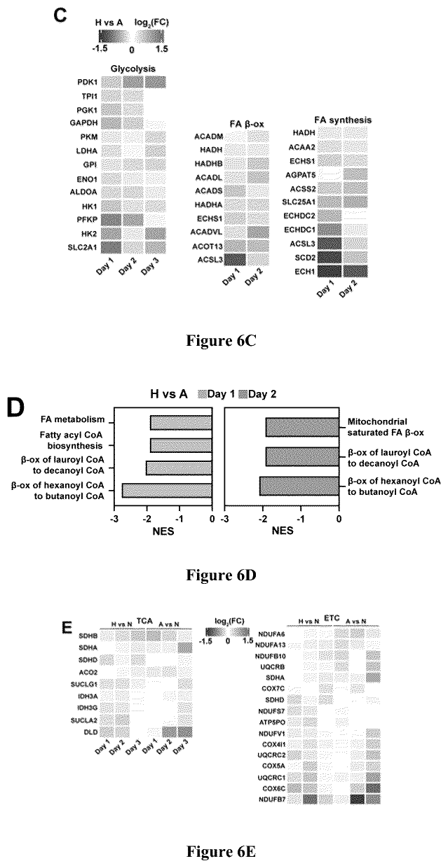

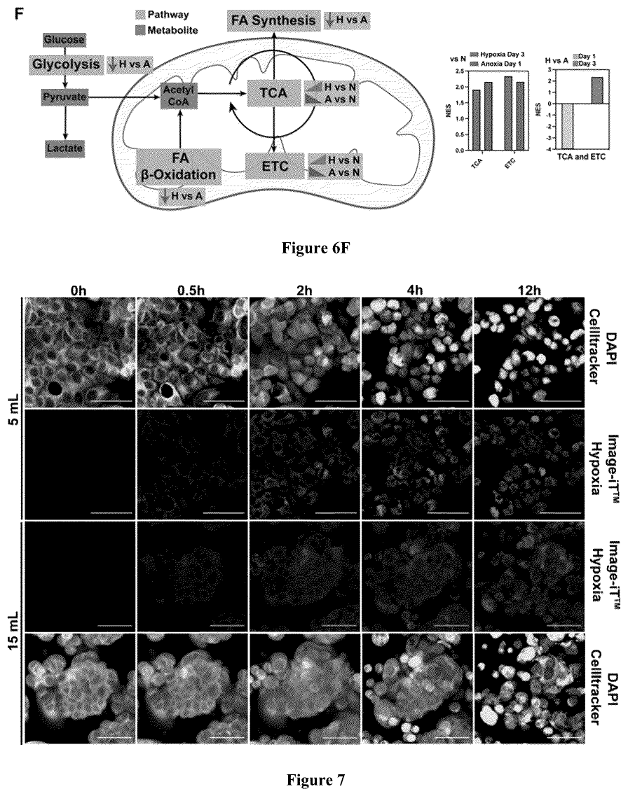

To further characterize low O₂ responses in 4T1 cells, we explored protein synthesis, hypoxic responses, and metabolic reprogramming at the pathway and protein level. As expected, RNA processing and protein translation pathways were downregulated in hypoxia and anoxia compared to nomoxia (FIG. 6A). Most of these pathways were upregulated in hypoxia compared to anoxia, suggesting downregulation as a function of O₂ tension. Surprisingly, translation elongation was upregulated in anoxia compared to hypoxia after 1 day of culture, suggesting distinct regulation in the acute anoxic response.

Next, we looked at pathways associated with the hypoxic response, and discovered that hypoxia and reactive O₂ species (ROS) pathways were upregulated in anoxia and EMT was upregulated in hypoxia (FIG. 6B). Proteins associated with hypoxia-induced stress responses showed higher abundance in anoxia for all three days. In addition, proteins associated with tumor progression, invasion, and metastasis, were upregulated in hypoxia for the duration of the experiment.

FIG. 6C illustrates that 9 out of 10 enzymes involved in glycolysis are more abundant in anoxia compared to hypoxia. In addition, proteins involved in hypoxia-mediated metabolic reprogramming show increased abundance in anoxia. We also examined fatty acid β-oxidation and synthesis, which are downregulated and upregulated in hypoxia respectively. Interestingly, PSEA suggests that both of these processes are downregulated in anoxia compared to hypoxia for the first 2 days of culture (FIG. 6D). Most enzymes involved in mitochondrial acyl-CoA to acetyl-CoA conversion (i.e., fatty acid β-oxidation) were upregulated in anoxia.

Finally, we examined oxidative phosphorylation, which is expected to decrease in low O₂ tensions. Unexpectedly, for hypoxia vs. anoxia, the tricarboxylic acid (TCA) cycle and electron transport chain (ETC) processes were downregulated after 1 day of culture and upregulated after 3 days of culture (FIG. 6E). Clustering of TCA and ETC enzymes suggests increasing oxidative phosphorylation activity in hypoxia, yet decreasing activity in anoxia (compared to normoxia). Taken together, these results suggest that 4T1 cells have distinct hypoxic and anoxic metabolic responses (FIG. 6F).

### INCORPORATION BY REFERENCE

All publications, US patents, and US and PCT published patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of culturing cells, comprising:
a. providing the cells and a culture incubator comprising a cell culture container; and an inflow of gas, wherein the cells are contained in the cell culture container;
b. providing a continuous inflow of a cell culture gas to the culture incubator;
c. incubating the cells in the culture incubator;
d. monitoring the pericellular oxygen concentration of the cells in the cell culture container; and
e. adjusting the inflow of the cell culture gas, thereby adjusting the pericellular oxygen concentration to a target oxygen concentration.

2. The method of claim 1, wherein the cell culture container further comprises an oxygen sensor for measuring the pericellular oxygen concentration.

3. The method of claim 2, wherein the oxygen sensor is located at the bottom of the cell culture container.

4. The method of claim 1, wherein the pericellular oxygen concentration is measured using a mathematically modeled equation that predicts pericellular oxygen tension in an oxygen-controlled cell culture.

5. The method of claim 4, wherein the mathematically modeled equation is Michaelis-Menten kinetics.

6. The method of claim 1, wherein the cell culture gas is nitrogen, argon, carbon dioxide, oxygen, and/or ambient air.

7. (canceled)

8. The method of claim 1, wherein the culture incubator comprises a humidity sensor, a carbon dioxide sensor, a temperature sensor, and/or an oxygen sensor.

9. The method of claim 1, wherein the culture incubator further comprises an inlet with a mass flow controller for carbon dioxide and/or a mass flow controller for nitrogen.

10. The method of claim 1, wherein the culture incubator further comprises an outlet that allows ambient air into the incubator.

11. The method of claim 1, wherein the cells are eukaryotic cells or prokaryotic cells.

12. The method of claim 11, wherein the cells are eukaryotic cells; and the eukaryotic cells are cell strains derived from an animal, a plant, or an insect, a primary culture product, or a fungus.

13. The method of claim 11, wherein the cells are prokaryotic cells; and the prokaryotic cells are bacteria including Escherichia coli, Bacillus subtilis, cyanobacteria, actinomycetes methanogen, extreme halophile, or hyperthermophile.

14. The method of claim 1, wherein the target oxygen concentration is for anoxic conditions.

15. The method of claim 14, wherein the target oxygen concentration is 0% to about 0.5% oxygen.

16. (canceled)

17. The method of claim 1, wherein the target oxygen concentration is for hypoxic conditions.

18. The method of claim 17, wherein the target oxygen concentration is about 0.5% to about 1% oxygen.

19. (canceled)

20. The method of claim 1, wherein the target oxygen concentration is for physioxic conditions.

21. The method of claim 20, wherein the target oxygen concentration is about 2% to about 5% oxygen.

22. (canceled)

23. The method of claim 1, wherein the culture incubator is a bioreactor.

24. (canceled)

25. A culture incubator, comprising
a. a cell culture container; and an inflow of gas, wherein cells are contained in the cell culture container; and
b. an oxygen sensor to monitor the pericellular oxygen concentration of the cells in the cell culture container;
wherein the pericellular oxygen concentration is adjusted to a target oxygen concentration by adjusting the inflow of a cell culture gas.

26-46. (canceled)
